(19) United States
(12) Patent Application Publication
(10) Pub. No.: US 2019/0125288 A1
(43) Pub. Date: May 2, 2019

(54) DIAGNOSIS AND PROGNOSIS OF MEDICAL CONDITIONS USING CRIBRIFORM PLATE MORPHOLOGY

(71) Applicant: Leucadia Therapeutics, LLC, Riverside, CA (US)

(72) Inventor: Douglas Wayne Ethell, Riverside, CA (US)

(21) Appl. No.: 16/175,623

(22) Filed: Oct. 30, 2018

Related U.S. Application Data
(60) Provisional application No. 62/579,063, filed on Oct. 30, 2017.

(51) Int. Cl. A61B 6/00; A61K 31/445; A61K 31/27; A61K 31/13; A61K 31/55; A61K 31/498; A61B 5/00; A61B 5/055; A61B 6/03; A61B 8/08

(52) U.S. Cl. CPC A61B 6/5217; A61B 5/7282; A61K 31/27; A61K 31/13; A61K 31/55; A61K 31/498; A61B 5/0059; A61B 5/055; A61B 5/4088; A61B 5/4538; A61B 6/032; A61B 6/037; A61B 8/0875; A61B 5/4839; A61B 8/5223; A61B 5/7275; A61K 31/445

(57) ABSTRACT

Methods of diagnosis, prognosis, and treatment of a neurological disease using cribriform plate morphology are provided.

### CROSS-REFERENCE TO RELATED APPLICATIONS

This application claims priority to U.S. Application No. 62/579,063 filed Oct. 30, 2017, the disclosure of which is incorporated by reference herein in its entirety.

### BACKGROUND

Alzheimer's disease (AD) is a neurodegenerative brain disorder and a common cause of dementia in the elderly that affected over 5.4 million people in the United States of America in 2016. Since its first description by Alois Alzheimer in 1907 this disease has been thought to result from pathological features in the brain called plaques and tangles. Many of clinical efforts to stop or prevent the progression of Alzheimer's Disease-related pathology have failed. Thus, there are urgent needs of novel compositions, tools, systems and/or methods to provide improved treatment of Alzheimer's Disease.

### BRIEF SUMMARY OF THE INVENTION

In one aspect, provided herein is a method of detecting an occluded aperture in a cribriform plate of a subject. The method may include (i) imaging the cribriform plate of a subject thereby forming a cribriform plate image and (ii) identifying an occluded aperture based on the cribriform plate image, thereby detecting an occluded aperture in the cribriform plate of the subject.

In another aspect, provided herein is a method of diagnosing a subject suspected of having a neurological disease. The method may include (i) imaging the cribriform plate of a subject thereby forming a cribriform plate image and (ii) determining whether the cribriform plate has an occluded aperture based on the cribriform plate image, wherein the presence of the occluded aperture indicates the subject has the neurological disease

In another aspect, provided herein is a method of treating a neurological disease in a subject. The method may include (i) imaging the cribriform plate of a subject thereby forming a cribriform plate image, (ii) identifying an occluded aperture based on the cribriform plate image, thereby detecting an occluded aperture in a cribriform plate of a subject and (iii) draining cerebrospinal fluid from an extracellular compartment above the cribriform plate of the subject.

In another aspect, provide herein is a system including at least one data processor and at least one memory including instructions. At least one data processor may execute the instructions to result in operations including (i) storing, in a database, a first model of a cribriform plate at a first stage of developing a neurological disease, (ii) querying the database to determine whether the first model matches a second model of a cribriform plate of a first subject and (iii) determining, based at least on the first model being matched to the second model, whether the first subject has the neurological disease.

### BRIEF DESCRIPTION OF THE DRAWINGS

FIG. 1 shows a reference micro-computed tomography (microCT) image of a human cribriform plate with labeling of crista galli, lateral walls, anterior wall, posterior wall, midline base of the crista galli, floor of the olfactory fossa or cribriform plate and several apertures. The perspective of the sample is superior to the cribriform plate where the frontal lobes would be located. Arrowheads near the top of the image indicate direction toward the front of the skull.

FIGS. 2A-2C show high-resolution microCT images of human cribriform plate from a post-mortem 26-year-old (26 y) woman. Images show different perspectives of the same structure from above (FIG. 2A), below (FIG. 2B) and an oblique front angle (FIG. 2C). These figures illustrate the delicate nature of cribriform plate bone structure in early adult life with numerous apertures created by thin bridges of bone. Along the base of the crista galli several large apertures straddle and impinge the midline. At the posterior-end large apertures extend into the back wall nearly horizontal to the floor of the cribriform plate. In the anterior-most portion of the olfactory fossa elongated apertures angle forward-back on either side of the crista galli.

FIGS. 3A-3C show high-resolution microCT images of human cribriform plate from a 75-year-old (75 y) woman who died from complications of Alzheimer's Disease. A diagnosis of Alzheimer's Disease was confirmed post-mortem using standard pathological and histological features that were consistent with the disease. Images shown are from above (FIG. 3A), below (FIG. 3B) and an oblique front angle (FIG. 3C) comparable to FIGS. 2A-2C. These images illustrate bone thickening and ossification of the cribriform plate that occurs in older individuals, particularly Alzheimer's Disease patients. Areas containing a delicate web of apertures and bone in the younger woman in FIG. 2 are occupied by thickened arches and bony plates that occlude small apertures leaving only a few large apertures in this 75 y woman's cribriform plate. The posterior portion of the cribriform plate largely includes a thick bony veil that descends from the top to the floor of the olfactory fossa as indicated with arrows. Anterior to this bony veil a flat region of the cribriform plate shows bilateral ossification, indicated with arrowheads.

FIGS. 4A-4C shows rendered images of cribriform plates in coronal section from two normal control subjects (labeled as control in the figure) and one Alzheimer's Disease subject. Samples were isolated from the skull of post-mortem subjects, counterstained with metallic iodine and scanned with a high-resolution microCT system. FIG. 4A is a sample from a 65 y male with no signs of dementia. Arrows are provided to indicate olfactory fossa width measures in each sample. Cerebrospinal fluid (CSF) conduits in the midline bone are indicated with an arrow and label. One aperture is also indicated with an arrow and label. FIG. 4B is a sample isolated from a 67 y male who died with no signs of dementia. FIG. 4C is a sample from a 79 y male who died with dementia and post-mortem pathology confirmed clinical signs of Alzheimer's Disease. A midline aperture is indicated in the Alzheimer's Disease sample but no cerebrospinal fluid conduits are visible. Nasal epithelium is indicated with arrows and label for the center and right samples. Widths and depth of the bony olfactory fossa (OF) are indicated with double arrow lines and labels. The center and right samples contain remnants of the olfactory bulb tissue above the cribriform plates as indicated with arrows and label.

FIGS. 5A-5G shows rendered images of cerebrospinal fluid (CSF) conduits in the cribriform plate and adjacent bones of controls (labeled as FIGS. 5A-5B) and five Alzheimer's Disease subjects labeled as FIGS. 5C-5G. Structures of cerebrospinal fluid conduits for each subject are shown from above the center of the olfactory fossa. Bone and other structures have been digitally removed. Below each image is the subjects' classification as normal control or Alzheimer's Disease, the age and sex of the subject, fixed brain weight, and ratings for degree of atrophy, atherosclerosis and ventricular size.

FIGS. 6A-6B show rendered models of human cribriform plate showing sagittal sections through apertures in a normal control subject (FIG. 6A) and an Alzheimer's Disease subject (FIG. 6B). MicroCT imaging was performed on excised cribriform plates isolated from a normal control subject and a subject with Alzheimer's Disease that was confirmed by post-mortem analysis of brain pathology. The top rendering shows the normal control subject with labels to indicate bone of the cribriform plate, bundles of CN1 nerve fibers, subarachnoid evaginations and cerebrospinal fluid conduits at top of the cribriform plate that has been digitally removed. The lower rendering is from an Alzheimer's Disease subject. The girth and complexity of CN1 nerve bundles is greater in normal control subjects than in Alzheimer's Disease subjects, which is reflected by a paucity of small apertures and narrowing of large apertures in the cribriform plate.

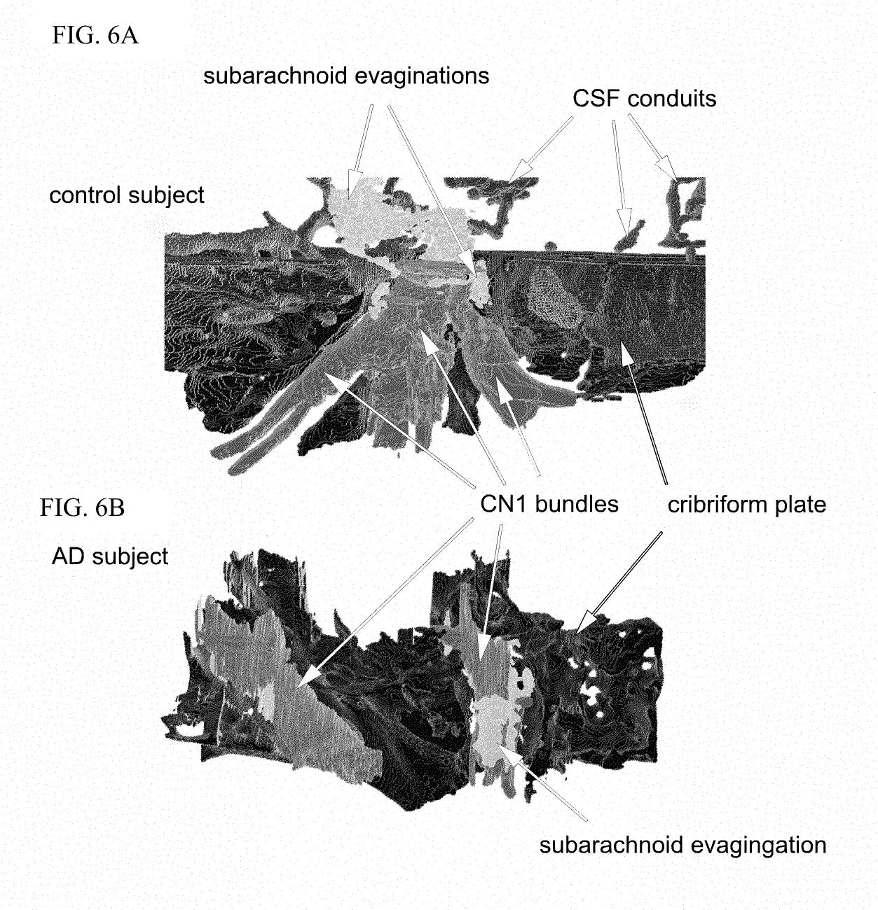

FIGS. 7A-7C illustrate how a CT scan is converted into a 3D model that allows text-based vector position information to be exported into a geometric positional map file (GPMF). In FIG. 7A, CT information has been rendered into a gray-scale image. The white outlining in FIG. 7B highlights three midline apertures. A digital 3D model is produced that represents the cribriform plate as a multi-faceted polygon, often exceeds 10,000 sides. FIG. 7B shows the region highlighted from FIG. 7A as it appears in a 3D model. Bright vector spots highlight the contours of the apertures. Cartesian information on the location of each vector is exported to a table as shown in FIG. 7C. Vector locations in regions surrounding all apertures processsed and exported into GPMF, which is used in the processing pipeline of embodiments. Further landmarking provides a geometric pattern of other structures such as olfactory fossa dimensions, crista galli, lateral, posterior and anterior walls. Computational analysis utilizes geometric morphology, linear discriminant analysis (LDA) and principal component analysis (PCA) to compare aperture sizes and positions in a patient's scan to corresponding markers in a database of known cases. In embodiments, based on comparisons of cribriform plate morphology in that patient and similar cases in the database, the processing pipeline calculates total aperture openings to determine a probability score that can be used in the diagnosis of and treatment of patients in need.

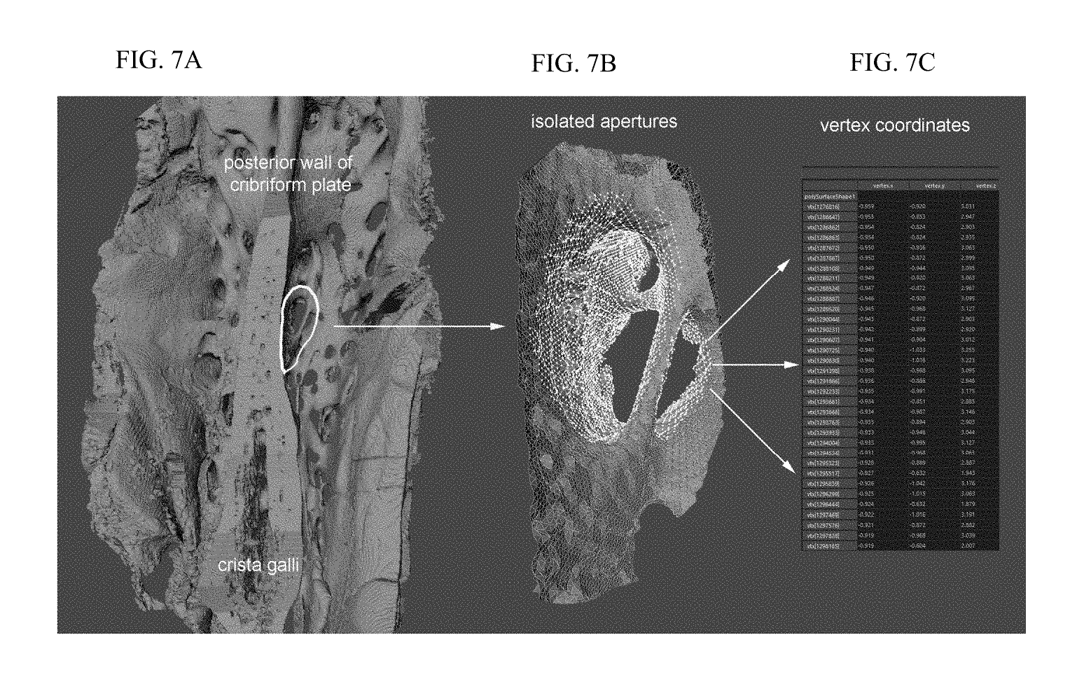

FIG. 8 shows a flow diagram to process a sample or subject for a high-resolution reference database of cribriform plate morphology. Steps are numbered for identification. (1) A head CT scan can be collected from a post-mortem subject using a clinical grade scanner. (2) The clinical grade scan can be imported into image analysis program and process to generate a digital 3D model. (3) Geometric landmarks in and around the cribriform plate can be noted and output as a geometric positional map file (GPMF). (4) The cribriform plate and surrounding bone can be carefully removed from the skull and (5) scanned with a high-resolution microCT scanner. (6) A digital model can be made from the microCT scan and (7) geometric landmarks can be recorded for output as a GPMF. (8) GFMPs from the clinical and microCT scans can be combined using geometric morphometry and differences can be reconciled to produce a GPMF that is a best fit for both. (9) Isolate cribriform plate sample can be stained with a contrasting agent such as metallic-iodine. (10) The contrast-enhanced sample can be scanned with microCT. (11) The scan can be imported into an image analysis program, and segmented into bone, blood vessels, CN1 nerves, cerebrospinal fluid flow channels and cerebrospinal fluid conduits. (12) The location of each aperture can be defined by GPMFs and the corresponding cross-sectional area of cerebrospinal fluid flow channels can be used to calculate cerebrospinal fluid flow capacity (CFC) for each aperture. (13) GPMFs and CFC values can be exported to the theoretical cerebrospinal fluid (tCFC) workflow detailed in FIG. 9. (14) Volumes of cerebrospinal fluid conduits in the base of the crista galli and other regions of the sample can be calculated using segmented data that are correlated with (15) local bone thickness calculate relative values in those areas. (16) Cerebrospinal fluid conduit correlations can be exported to the cerebrospinal fluid conduit volume workflow. (17) Values for the cross-sectional area of all CN1 fibers can be kept for use in olfactory capacity and discrimination calculations.

FIG. 9 shows a flow diagram for the generation of theoretical cerebrospinal fluid (tCSF) flow capacity in a patient. Steps are indicated with numbers for clarity. (1) A clinical CT scan can be imported that includes the cribriform plate and surrounding areas. (2) Areas outside the region of interest (ROI) can be cropped out to reduce file size. (3) Image analysis software can be used to generate a digital 3D model of the ROI. (4) Geometric landmarks can be made for surface topology, and unusual features can be noted such as gross asymmetry. (5) Geometric landmarks can be output in a geometric positional map file (GPMF). (6) Geometric morphometry can be used to align the patient's GPMF with (7) GPMFs in a high-resolution database of cribriform plate morphology, using regional similarities to identify homologous regions from one to many reference cases. (8) Multivariate analysis (e.g. PCA and LDA) can be used to sort the top alignments from each region of the cribriform plate. (9) Matching regions with highest statistical significance can be superimposed on the digital 3D model made from the patient's clinical CT scan. (10) Theoretical cerebrospinal fluid flow capacity for the cribriform plate can be calculated from cerebrospinal fluid channels for each region, using data in the database, described in FIG. 8.

FIG. 10 depicts a system diagram illustrating a system for assessing human cribriform plate morphology, in accordance with some example embodiments.

FIG. 11 depicts a flowchart illustrating a process for assessing human cribriform plate morphology, in accordance with some example embodiments.

FIG. 12 depicts a block diagram illustrating a computing system, in accordance with some example embodiments.

### DETAILED DESCRIPTION

The present disclosure relates to, inter alia, a procedure and system to analyze cribriform plate morphology as a diagnostic and prognostic indicator for neurological diseases such as Alzheimer's disease.

### Definitions

Unless defined otherwise, all technical and scientific terms used herein have the same meaning as commonly understood by one of ordinary skill in the art to which the disclosures belong. All publications mentioned herein are incorporated by reference to disclose and describe the methods and/or materials in connection with which the publications are cited.

Unless the context indicates otherwise, it is specifically intended that the various features of the disclosures described herein can be used in any combination. Moreover, the disclosure also contemplates that in some embodiments any feature or combination of features set forth herein can be excluded or omitted. To illustrate, if the specification states that a complex has components A, B and C, it is specifically intended that any of A, B or C, or a combination thereof, can be omitted and disclaimed singularly or in any combination.

The term “about” means a range of values including the specified value, which a person of ordinary skill in the art would consider reasonably similar to the specified value. In embodiments, “about” means within a standard deviation using measurements generally acceptable in the art. In embodiments, about means a range extending to +/−10% of the specified value. In embodiments, about means the specified value.

Also as used herein, “and/or” refers to and encompasses any-and-all possible combinations of one or more of the associated listed items.

It must be noted that as used herein, the singular forms “a”, “an”, and “the” include plural referents unless the context clearly dictates otherwise.

As used herein, the term “comprising” is intended to mean that the compositions and methods include the recited elements, but do not exclude others. As used herein, the transitional phrase “consisting essentially of” (and grammatical variants) is to be interpreted as encompassing the recited materials or steps “and those that do not materially affect the basic and novel characteristic(s)” of the recited embodiment. Thus, the term “consisting essentially of” as used herein should not be interpreted as equivalent to “comprising.” “Consisting of” shall mean excluding more than trace elements of other ingredients and substantial method steps for administering the compositions disclosed herein. Aspects defined by each of these transition terms are within the scope of the present disclosure.

The terms or “acceptable,” “effective,” or “sufficient” when used to describe the selection of any components, ranges, dose forms, etc. disclosed herein intend that the component, range, dose form, etc. is suitable for the disclosed purpose.

The term “neurological disease” as used herein, refer to any disorder, disease, condition and/or syndrome due to or resulting from neurologic origin. The term “neurological disease” includes neuroglogical diseases, psychiatric diseases, psychological diseases, and/or cerebrovascular diseases, or other diseases or disorders having nuerogological symptomology or neurological origin. The term “neurological disease” is equivalent to “neuroglogical disease and/or psychiatric disease” and “neurological disease and/or psychiatric disorder.” Neurological diseases include, but are not limited to, Alzheimer's disease (AD), Down syndrome, Parkinson's disease, Pick's disease, dementia, brain injury (e.g., concussive brain injury), supranuclear palsy, Creutzfeldt-Jakob disease, normal pressure hydrocephalus, multiple sclerosis, Huntington's Disease, amyotrophic lateral sclerosis (ALS), muscular dystrophy, epilepsy, autism, cerebral palsy, or any combination thereof. Dementia includes, but is not limited to frontotemporal dementia, mild cognitive impairment, idiopathic dementia, vascular dementia, and the like. In aspects, the neurological disease is Alzheimer's disease. In aspects, the neurological disease is Down syndrome. In aspects, the neurological disease is Parkinson's disease. In aspects, the neurological disease is Pick's disease. In aspects, the neurological disease is brain injury. In aspects, the neurological disease is a concussive brain injury. In aspects, the neurological disease is supranuclear palsy. In aspects, the neurological disease is Creutzfeldt-Jakob disease. In aspects, the neurological disease is normal pressure hydrocephalus. In aspects, the neurological disease is multiple sclerosis. In aspects, the neurological disease is Huntington's Disease. In aspects, the neurological disease is amyotrophic lateral sclerosis. In aspects, the neurological disease is muscular dystrophy. In aspects, the neurological disease is epilepsy. In aspects, the neurological disease is autism. In aspects, the neurological disease is cerebral palsy. In aspects, the neurological disease is dementia. In aspects, the neurological disease is frontotemporal dementia. In aspects, the neurological disease is mild cognitive impairment. In aspects, the neurological disease is idiopathic dementia. In aspects, the neurological disease is vascular dementia.

The term “central nervous system” (CNS), as used herein, refers to brain, spinal cord, optic, olfactory, and auditory systems. The CNS includes both neurons and glial cells (neuroglia), which are support cells that aid the function of neurons.

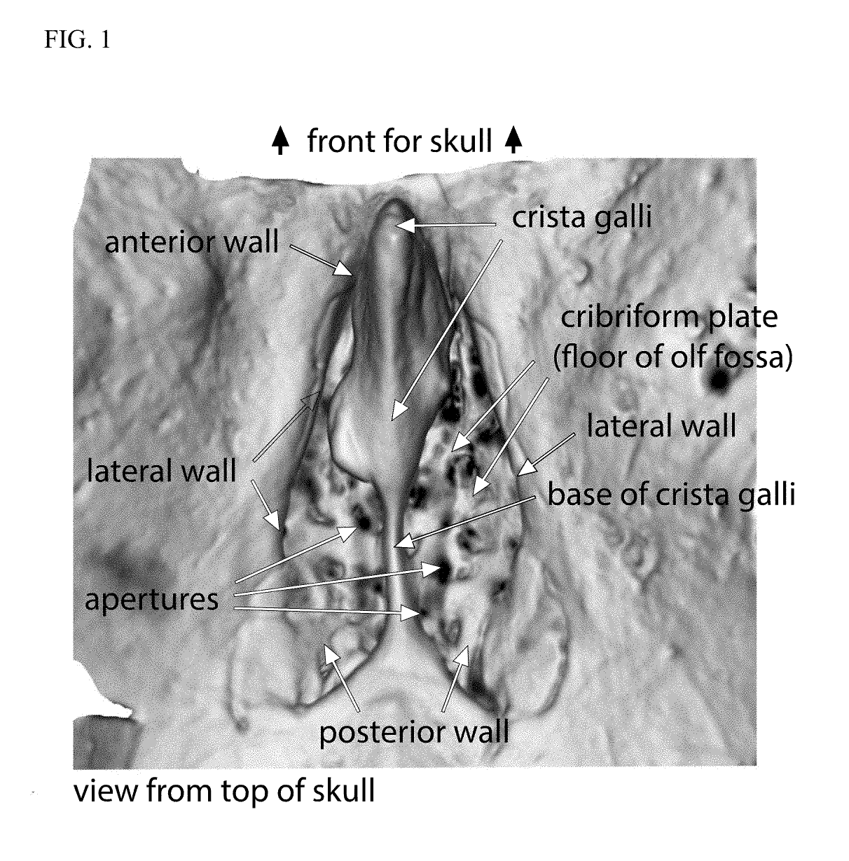

The term “cribriform plate,” as used herein, in the context of human anatomy, refers to a generally horizontal plate of an ethmoid bone perforated with numerous foramina for the passage of olfactory nerve filaments from a nasal cavity. The cribriform plate as described here forms the floor and the posterior wall of the olfactory fossa as well as inferior portions of medial walls and laterals walls in addition the medial structures that are continuous with the crista galli. A typical structure of cribriform plate is illustrated in FIG. 1 and described as follows: Projecting upward from the middle line of the plate is a thick, smooth, bulbous process, the crista galli. The long thin posterior border of the crista galli serves for the attachment of the falx cerebri. Its anterior border, short and thick, articulates with the frontal bone, and presents two small projecting alae (wings), which are received into corresponding depressions in the frontal bone and complete the foramen cecum. Its sides are generally smooth, and sometimes bulging from presence of a small air sinus in the interior. On either side of the crista galli, the cribriform plate can be narrow and deeply grooved; it can support the olfactory bulb and can be perforated by foramina for the passage of the olfactory nerves and blood vessels. The foramina in the middle of the groove can be small and transmit the nerves through the roof of the nasal cavity; those at the medial and lateral parts of the groove can be larger—the former can transmit the nerves to the upper part of the nasal septum, the latter those to the superior nasal concha. At the front part of the cribriform plate, on either side of the crista galli, can be a small fissure that is occupied by a process of dura mater. Lateral to this fissure is a notch or foramen which can transmits the nasociliary nerve; from this notch a groove extends backward to the anterior ethmoidal foramen. An olfactory fossa refers to a bone structure that is formed by a horizontal lamella of a cribriform plate, its vertical lamellae and a part of the orbital plate of the frontal bone. The depth of the olfactory fossa varies and has been classified by Keros into three types: type 1: about 1-about 3 mm, type 2: about 4-about 7 mm, type 3: about 8-about 17 mm.

The terms “cerebral spinal fluid,” “cerebrospinal fluid” or “CSF” refer to a clear, colorless body fluid found in the brain and spinal cord. It is produced in the choroid plexuses of the ventricles of the brain, and absorbed in the arachnoid granulations. In general, there is about 125 mL of cerebrospinal fluid at any one time, and about 500 mL is generated every day. Cerebrospinal fluid acts as a cushion or buffer for the brain, providing basic mechanical and immunological protection to the brain inside the skull. The cerebrospinal fluid also serves a vital function in cerebral autoregulation of cerebral blood flow.

The term “cerebral spinal fluid conduit(s)” refers to fluid-filled tunnel(s) that run(s) forward and back and sideways through bony structures of the cribriform plate, crista galli, surrounding ethmoid bone and posterior wall. In embodiments, conduits may be different from apertures because they may not form a clear connection between the olfactory fossa (above) and the sinus cavity (below).

The term “cerebral spinal fluid channel(s)” refer to fluid-filled space(s) within soft tissue of the olfactory bulbs and soft tissue that runs down the inside of apertures. In embodiments, the channels are how cerebral spinal fluid gets out of the olfactory fossa and eventually into the nasal submucosa.

The term “morphology” or “morphological feature(s),” as used herein, refers to one or more properties related to external structure. For example, in the context of “cribriform plate morphology” or “cribriform plate morphological feature(s)”, the term includes, but not limited to, an anterior wall dimension of the cribriform plate, a posterior wall dimension of the cribriform plate (the anterior being closer to the front side of a skull and the posterior being closer to the rear side of the skull when viewed from the top of the skull), a lateral boundary of an olfactory fossa, a length of an olfactory fossa, a width at four locations of an olfactory fossa, a depth of an olfactory fossa, an angle of the posterior wall to the floor of the cribriform plate, a size of each aperture, a branching pattern of apertures, a position of each aperture, a degree of bone ossification immediately around each aperture, four outer corners of the cribriform plate distance from a midline, margins of a crista galli, a size of a crista galli, or any combination thereof.

The term “imaging” as used herein refers to a process or action to make a visual representation of something, e.g. a body part by scanning it with a detector or electromagnetic beam device such as computed tomography (CT), magnetic resonance imaging (MRI), positron emission tomography (PET), ultrasound, or far-red imaging.

The terms “three dimensional image(s),” “three dimensional imaging,” “3D image(s)” or “3D imaging,” as used herein, refer to images having three dimensions. In particular, the images can provide the illustration of depth, width or varying distances of elements present in a subject of the images such that a pictorial representation of the subject on a two-dimensional medium can be provided. In the context of clinical imaging, which is performed for the medical purposes, such as diagnostic, prognostic and/or therapeutic purposes, three dimensional images can be obtained, for example, by one or more of computed tomography (CT), magnetic resonance imaging (MRI), positron emission tomography (PET), ultrasound, or far-red imaging.

The terms “individual” or “subject” are used interchangeably herein and refer to any subject for whom diagnosis, treatment or therapy is desired. In embodiments, the subject is a mammal. In some aspects, the subject is a human being. In embodiments, the subject is a human patient. In embodiments, the subject has or is suspected of having one or more symptoms of neurological disease. In embodiments, the subject is a human who is diagnosed with a risk of neurological disease at the time of diagnosis or later. In embodiments, the subject is a human who is not a patient of neurological disease nor diagnosed with a risk of the disease but merely desires to have or benefits from a diagnosis of the disease.

The terms “treatment,” “treating” or “treat,” as used herein, refer to acting upon a disease, disorder, or condition with an agent to reduce or ameliorate harmful or any other undesired effects of the disease, disorder, or condition and/or its symptoms. “Treatment,” as used herein, covers the treatment of a subject in need thereof, and includes treatment of a neurological disease. “Treating” or “treatment of” a condition or subject in need thereof refers to (1) taking steps to obtain beneficial or desired results, including clinical results such as the reduction of symptoms; (2) preventing the disease, for example, causing the clinical symptoms of the disease not to develop in a patient that may be predisposed to the disease but does not yet experience or display symptoms of the disease; (3) inhibiting the disease, for example, arresting or reducing the development of the disease or its clinical symptoms; (4) relieving the disease, for example, causing regression of the disease or its clinical symptoms; or (5) delaying the disease.

The terms “control,” “control sample,” “control image,” “reference,” “reference sample” or “reference image” as used herein, refer to a subject, sample or image that serves as a reference, usually a known reference, for comparison to a test subject, a test sample or test image. In embodiments, the control or reference may be two types, a disease control (or disease-associated control) and a normal (non-disease control). In embodiments, a control, in particular a disease control (or reference) is a control subject, sample or value taken from a patient who was previously diagnosed with a neurological disease of interest or any population thereof. In embodiments, a disease control is a control subject, sample, image or value taken from a subject who was previously known to have symptoms that are indicative of or associated (i.e. known to be present) with a neurological disease of interest or any population thereof. In the cases concerning a certain neurological disease, the symptoms present in the neurological disease control can include any morphological feature(s) of cribriform plate that are indicative of or associated with the concerned neurological disease. In embodiments, a normal (non-disease) control refers to a subject, sample, image or value taken from a health subject who is known not to have or suspected of not having a neurological disease. In embodiments, a control or reference can also represent an average value gathered from a population of similar individuals, e.g., neurological and/or psychiatric patients or healthy individuals with a similar medical background, same age, weight, etc. In embodiments, a control or reference image can also be obtained from the same individual, e.g., from an earlier-obtained sample, prior to disease, or prior to treatment, especially when a plurality of images from the same individual are monitored over a course of time.

The term “therapeutic intervention,” as used herein, refers to medicine (e.g. therapeutic compositions) and/or therapy (e.g. chemical and/or surgical procedures) used to reduce or cure disease or pain by the involvement and intercession of therapeutic practice. Therapeutic intervention can vary in methods, for example, depending on a condition or disease of a patient who is in need of such a therapeutic intervention. In the context of a method treating a neurological disease in a subject, a therapeutic intervention can be performed to a subject identified for the treatment. In such a context, the therapeutic intervention can contain a step of perforating a cribriform plate of the subject, so as to drain at least part of cerebrospinal fluid and/or increase the cerebrospinal fluid conduit. The term “perforate,” “perforating” or “perforation” of a cribriform plate can refer to (1) increase of a number of apertures on the cribriform plate and/or (2) increase of a size, area and/or depth of one or more aperture on the cribriform plate.

The terms “administration,” “administering” and the like refer both to direct administration, which may be administration to cells in vitro, administration to cells in vivo, administration to a subject by a medical professional or by self-administration by the subject and/or to indirect administration, which may be the act of prescribing a composition. Typically, an effective amount is administered, which amount can be determined by one of skill in the art. Any method of administration may be used. Compounds (e.g., drugs) can be administered to a subject. Administration to a subject can be achieved by, for example, oral delivery, intravascular injection, direct intratumoral delivery, and the like.

It is understood that the examples and embodiments described herein are for illustrative purposes only and that various modifications or changes in light thereof will be suggested to persons skilled in the art and are to be included within the spirit and purview of this application and scope of the appended claims.

Cribriform Plate Morphology and its Implication in Neurological Disease

Cribriform plate apertures contain conduits for cerebrospinal fluid flow from the olfactory fossa to nasal submucosa. Therefore, reductions in the number, size, location and total cross-sectional area of these apertures may impact cerebrospinal fluid outflow capacity (CFC) of the cribriform plate. The disclosure provided herein relates to the inventor's establishment that a neurological disease such as Alzheimer's Disease is associated with certain morphological changes in the cribriform plate morphology that may impact the flow of cerebrospinal fluid from soft tissues, for example, in the olfactory fossa to nasal submucosa. Therefore, in embodiments accurate assessment of this capacity is needed for the diagnosis and treatment of patients who may suffer from neurological diseases related to reduced cerebrospinal fluid-mediated clearance of upstream brain regions, including Alzheimer's Disease.

Although the pathological progression of Alzheimer's Disease varies from person to person, neuritic plaques and neurofibrillary tangles may occur first in the medial temporal gyms (MTG), a region that is phylogenetically older (allocortex) than neocortex. While interstitial spaces in both regions may be cleared by cerebrospinal fluid from the lateral ventricle, the pattern of cerebrospinal fluid efflux can be different. In the neocortex, cerebrospinal fluid in the lateral ventricles may flow outward through interstitial spaces of the neocortex toward subarachnoid spaces on the surface of the forebrain. Some of the cerebrospinal fluid that emerges may be resorbed by arachnoid granulations and glymphatics, but the remainder may flow posteriorly past the cerebellum and down sub-arachnoid spaces that surround the spinal cord. As cerebrospinal fluid flows past the brainstem and spinal cord, some of it may leak out of the CNS along sheaths that cover cranial nerves and spinal nerves along the way. A relatively small proportion of cerebrospinal fluid that emerges in sub-arachnoid spaces covering the neocortex may make it all the way down to the lumbar enlargement, where it can be collected by lumbar puncture, also known as a spinal tap. Ironically, the ease with which fluid can be collected by lumbar puncture has resulted in a prevailing view that all cerebrospinal fluid in the brain is the same.

Numerous searches for early biomarkers of Alzheimer's Disease used cerebrospinal fluid obtained from lumbar punctures, which resulted in variable and sometimes enigmatic results. Phylogenetically older regions of the forebrain cortex, known as allocortex include the hippocampal formation, entorhinal cortex, amygdala and piriform cortex of the MTG as well as the Nucleus basalis of Meynert (NbM) in the basal forebrain, which are the first regions of the brain to be affected by Alzheimer's Disease pathology. Cerebrospinal fluid that passes from the lateral ventricle into the allocortex does not drain along the same route as cerebrospinal fluid that enters the neocortex. Instead, cerebrospinal fluid in subarachnoid spaces of the MTG flows anteriorly toward the anterior pole where it runs into the loosely packed lateral olfactory stria and into the olfactory trigone immediately below the basal forebrain. cerebrospinal fluid flows from the inferior horn of the lateral ventricle into the alveus of the hippocampus and then percolates through the entorhinal cortex and surrounding areas. Along the way it picks up apoptotic debris and insoluble metabolites that are carried through interstitial spaces and perivascular spaces to subarachnoid spaces on the surface of the MTG. At the anterior pole of the MTG a rudimentary funnel channels cerebrospinal fluid into the lateral olfactory stria. Cerebrospinal fluid flows through channels in and around the lateral olfactory stria to the olfactory trigone, which lies immediately beneath the basal forebrain, a region that contains cholinergic neurons in the NbM that are critical for memory formation. Medial olfactory stria interconnect bilateral olfactory trigones. Cerebrospinal fluid that passes into the olfactory trigone proceeds anteriorly to along the olfactory tract to the olfactory bulb. Olfactory neurons in the nasal mucosa project axons upward in cranial nerve 1 (CN1) bundles that pass through the cribriform plate to the olfactory bulb above. As with cranial spinal nerves and other cranial nerves, cerebrospinal fluid from the olfactory bulb flows out of the CNS along CN1 bundles through discrete channels, described for the first time in this disclosure. Therefore, the cribriform plate can serve as the final exit for cerebrospinal fluid that clears interstitial spaces in the medial temporal lobe and the basal forebrain, two of the earliest regions affected by Alzheimer's disease pathology.

Disclosed herein are previously unknown cerebrospinal fluid flow channels and aspects of cribriform plate morphology. Factors that may impede cerebrospinal fluid flow along the olfactory stria, olfactory bulbs and across the cribriform plate may reduce the efficiency of interstitial clearance in upstream brain areas that include the basal forebrain and MTG. The cribriform plate of the ethmoid bone is typically considered a paired structure containing the floor and lower walls of the olfactory fossa. It separates olfactory bulbs above from nasal submucosa that lines the nasal cavity below. Provided herein are imaged and characterized bone and soft tissues in the cribriform plate to a level not previously reported. Using microCT to image cribriform plates excised from deceased subjects, it has been found herein that, in embodiments, the cribriform plate and crista galli may become ossified with age. The crista galli is a medial bone structure in the olfactory fossa that separates the olfactory fossa on each side. The crista galli typically projects upward past the olfactory fossa to provide anchor support for the falx cerebri, a thick connective tissue structure that connects to meninges of the cerebral hemispheres. Crista galli are generally large in older individuals than young subjects to the extent that it often spans the width of the olfactory fossa in subjects over age 85. Age-dependent enlargement of the crista galli may reduce the olfactory fossa and press downward on olfactory bulbs, which may constrict cerebrospinal fluid outflow. In embodiments, the width of the olfactory fossa becomes narrower in older subjects, due to medial displacement of the lateral walls, which may also compress olfactory bulbs.

The cribriform plate typically forms the floor of the olfactory fossa as well as the posterior wall the base of the crista galli and portions of the remaining walls. It is typically a porous bone with apertures between the olfactory fossa above and the nasal cavity below. Provided herein is an analysis of dried bone samples having thin and flat cribriform plates with semi-random patterns of apertures as typically seen in human skulls prepared for educational purposes. However, fresh specimens or those stored in saline solutions were observed to have retained a more sponge-like 3D morphology in which apertures can be seen to form tunnels at oblique angles to vertical. Importantly, historical texts and other descriptions of cribriform plate morphology have relied on dried bones and specimens where the 3D structure of the cribriform plate had been thinned and flattened, producing inaccurate accounts of this structure dating from Marcus Vitruvius Pollio (80-15 BCE) until the present time. Provided herein is information accumulated after scanning over 70 human cribriform plates with microCT. For example, it was found that the morphology of this structure is a more complex 3D structure than had been reported previously.

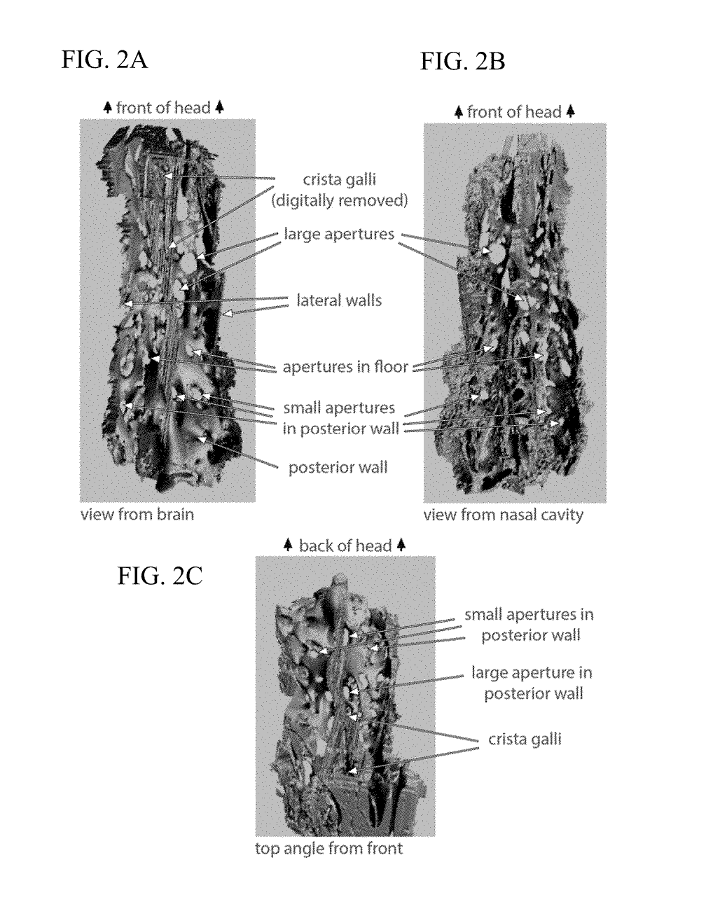

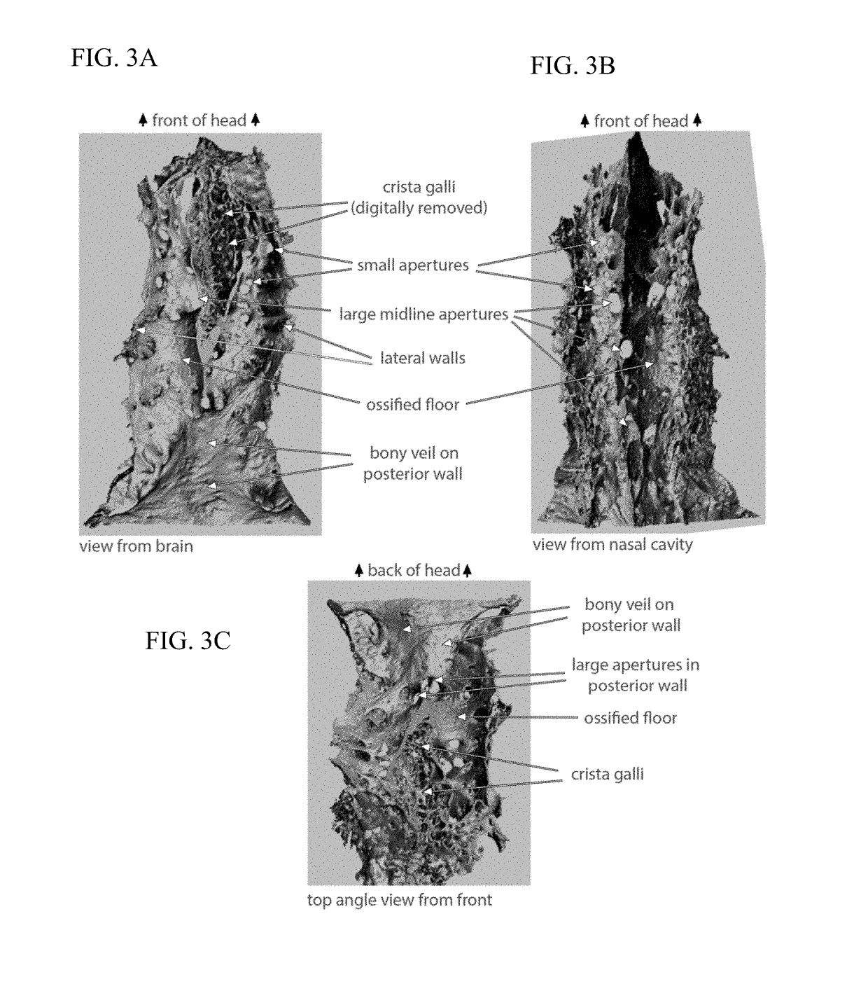

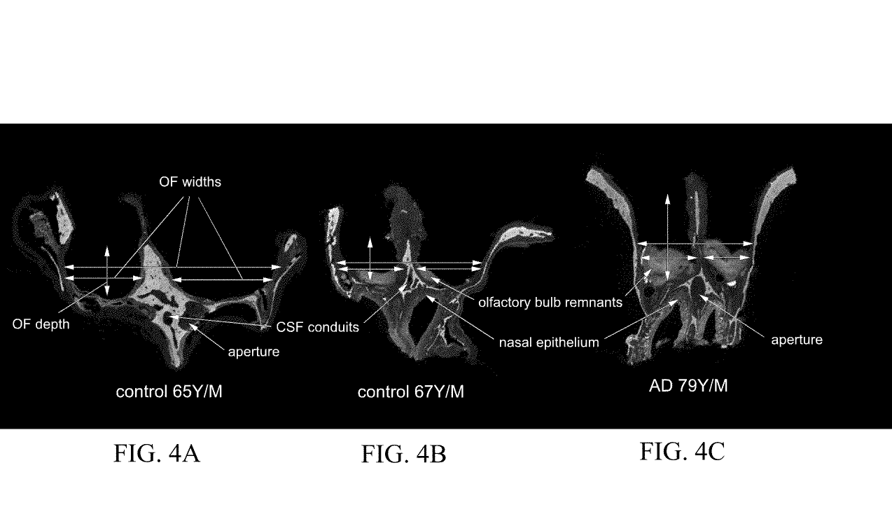

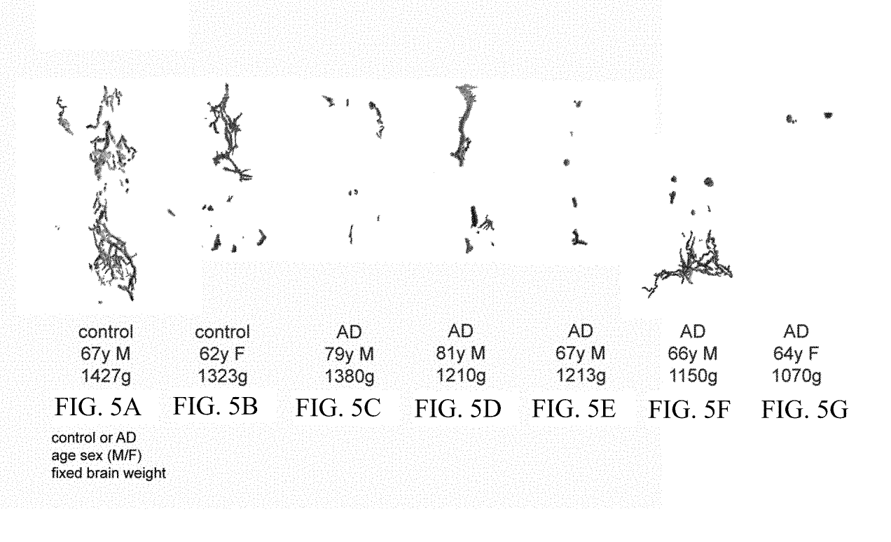

In embodiments, bony supports and the apertures that form between them form a web-like lattice in young people that may become ossified with age. Apertures along the midline and around the perimeter of the cribriform plate may run at angles to the vertical. For example, apertures at the posterior end of the olfactory fossa may run nearly horizontal and parallel to the floor of the cribriform plate as they project into the posterior wall (see FIG. 2 and FIG. 3). Apertures along the midline may run in a slanted anterior-posterior direction, while apertures along the lateral cribriform plate may angle away from the midline. Apertures within the cribriform plate may contain soft tissues that include olfactory nerve fibers (CN1) that pass from nasal epithelium (below) to olfactory bulbs (above). Contrast enhancement with metal-iodine before microCT imaging can provide differentiation of soft tissues down to resolutions of 8-16 microns in intact cribriform plates that had been excised from human skulls (see FIG. 4). Contrast enhancement can also allow resolution of olfactory nerve fibers (CN1) from other soft tissues within apertures of the cribriform plate and reveal fluid-filled conduits that run through these soft tissues. These fluid-filled channels may facilitate cerebrospinal fluid flow across the cribriform plate. Immediately below the cribriform plate, the nasal cavity may be lined with thick epithelial tissue that contains an extensive network of lymphatic vessels. Furthermore, bones of the crista galli, cribriform plate and surrounding bones may contain networks of fluid filled conduits that connect with subarachnoid invaginations, subarachnoid spaces in the olfactory fossa and nasal submucosa (see FIG. 5). These cerebrospinal fluid conduits were found more extensive in younger subjects and non-demented older subjects. Also, demented and Alzheimer's Disease subjects had diminished cerebrospinal fluid conduit networks within bones that make up the olfactory fossa. Therefore, these cerebrospinal fluid-containing conduits may serve to equilibrate cerebrospinal fluid flow and deficits in them may participate to the etiology of neurological diseases, including Alzheimer's Disease.

In addition, characteristic accumulations of amyloid-beta (Aβ) deposits in early Alzheimer's Disease can result from disruptions in interstitial fluid or cerebrospinal fluid flow that usually clears debris and toxins from extracellular spaces within phylogenetically older regions of the forebrain called allocortex. The earliest pathological features of Alzheimer's Disease may occur in allocortex and contiguous regions of the CNS. In homeostasis, cerebrospinal fluid may flow from the inferior horn of the lateral ventricle through interstitial spaces of the medial temporal gyms (MTG) into the lateral olfactory stria. Metabolite-laden cerebrospinal fluid may flow along the lateral olfactory stria to the olfactory trigone and along the olfactory tract to the olfactory bulb, where it may seep through the cribriform plate into nasal submucosa. Lymphatic vessels in the nasal submucosa may facilitate the removal of the cerebrospinal fluid and metabolites carried therein. The role of this cerebrospinal fluid flow route for neurological disease may relate to the clearance of metabolites, debris and toxins from interstitial spaces of the hippocampal formation, entorhinal cortex and other structures in the MTG, as well as the basal forebrain. Reductions in cerebrospinal fluid-mediated clearance of these structures could facilitate the accumulation of amyloid-beta (Aβ) in MTG, basal forebrain, and nearby areas, predisposing them to accumulate toxic metabolites that trigger the formation of pathological features including neuritic plaques and neurofibrillary tangles.

In embodiments, factors that reduce cerebrospinal fluid drainage across the cribriform plate can slow the clearance of cerebrospinal fluid from the MTG and basal forebrain. Other factors may include aging-related ossification of the skull in and around the cribriform plate, head trauma, broken nose, inflammation of the nasal epithelium, toxins that affect olfactory neuron survival and renewal, as well as vascular effects related to diabetes, obesity, and atherosclerosis—many of which have been proposed to affect Alzheimer's Disease risk. Reductions of cerebrospinal fluid-mediated clearance across the cribriform plate could also act synergistically with familial mutations linked to early-onset Alzheimer's Disease, including but not limited to PSEN1, PSEN2, APP. Higher ratios of longer and less soluble Aβ peptides may be produced in carriers of many mutations linked to early-onset Alzheimer's Disease. Longer Aβ species may be more prone to oligomerize and aggregate than shorter Aβ species, thereby reducing the age at which cribriform plate occlusion becomes clinically relevant.

Methods

In one aspect, the present disclosure provides diagnostic and therapeutic approaches for a neurological disease in a subject. In embodiments, the approaches of the disclosure are based on the assessment of cribriform plate morphology of the subject and comparison of the subject's cribriform plate morphology to references such as the cribriform plate imaging obtained from normal (non-disease) subjects and/or disease-associated subjects.

The cribriform plate may serve as the final outlet for cerebrospinal that clears toxins and metabolites from medial temporal gyms and basal forebrain, which are the first two regions of the brain to be affected by neuritic plaques and neurofibrillary tangles and in late-onset Alzheimer's Disease. Age-related ossification of the cribriform plate may occlude flow channels through which cerebrospinal fluid can pass out of the brain, and hence reduce the clearance of factors that trigger Alzheimer's Disease pathology. In embodiments the present disclosure provides methods and systems to assess cribriform plate morphology, including the presence and/or level of occluded aperture in the cribriform plate that may be used for the diagnosis and treatment of medical disorders. Thus, in embodiments the disclosure provides methods and systems to assess morphology of a human cribriform plate and surrounding bones as a prognostic and diagnostic indicator of neurological disease.

In embodiments, the disclosure provides a method of detecting an occluded aperture in a cribriform plate of a subject. The method may include imaging the cribriform plate of a subject, thereby forming a 3D model of the cribriform plate of the subject and (ii) identifying an occluded aperture based on the subject's cribriform plate image, thereby detecting an occluded aperture in the cribriform plate of the subject.

In embodiments, the disclosures provide a method to produce and analyze three-dimensional (3D) images of cribriform plates using scans. In embodiments, the scans can be obtained by clinical imaging methods, e.g. computed tomography (CT), magnetic resonance imaging (MRI), positron emission tomography (PET), ultrasound, far-red imaging. In embodiments, the cribriform plate of a subject is scanned a plurality of times, e.g. several times, several tens of times, several hundreds of times or several thousands of times. A number of data obtained from the plurality of times of scans can be processed to produce one or more of three-dimensional images of cribriform plate of the subject.

In embodiments, the disclosure provides a method to evaluate cribriform plate morphology such as porosity in a subject by producing a geomorphometric map. In embodiments, the method is performed by inputting medical imaging files of the cribriform plate, such as a head CT taken from the subject, to produce a 3D digital model of the cribriform plate and surrounding structures, including crista galli and ethmoid bone.

In embodiments, the identification of an occluded aperture based on the subject's cribriform plate image is done by comparing the subject's cribriform plate image to a cribriform plate control image. For example, the subject's geomorphometric map can be compared to cribriform plate images in a referential database that may have a plurality of control images (or data) from one or more normal (i.e. non-disease) subjects and/or one or more disease-associated subjects, i.e. a subject who were known to have a neurological disease or symptoms thereof. In embodiments, digital landmarks can be located on the 3D model and output to a geometric positional map file (GPMF). Landmarks from one scan can then be aligned with scans taken from the subject at a different time or with control subjects in database of GPMF's that are linked to medical records of other subjects, including those with confirmed diagnoses of a neurological disease such as Alzheimer's disease. In embodiments, one or more control geometric positional map files from the subjects who were confirmed with Alzheimer's Disease can form a reference database (or referential database). Thus, in embodiments the subject's GPMF can be compared to one or more control (or interchangeably referred to as one or more reference) GPMFs available from the database.

The comparison can be done using multivariate analyses, such a database of CP models using linear discriminant analysis (LDA) and principal component analysis (PCA). In embodiments, source images for the processing pipeline process may include scans of cribriform plates using imaging modalities, such as computed tomography (CT), magnetic resonance imaging (MRI), positron emission tomography (PET), ultrasound, far-red imaging. Therefore, in embodiments the cribriform plate image used in the method of the disclosure is a computed tomography (CT) cribriform plate image, magnetic resonance imaging (MRI) cribriform plate image, positron emission tomography (PET) cribriform plate image, ultrasound cribriform plate image, far-red imaging cribriform plate image or any combinations thereof.

In embodiments, the method includes a step of comparing one or more three-dimensional images of the cribriform plate obtained from a subject (i.e. the test subject) to one or more control or reference images, e.g. a plurality of three-dimensional images of the cribriform plate of one or more control subjects. In embodiments, the control or reference cribriform plate image is from a single control subject. In embodiments, the control or reference cribriform plate image is a composite image that is produced by imaging cribriform plates of a plurality of control or reference subjects. As further described below, the control or reference can be a normal (non-disease) control or a disease control (or interchangeably used with a disease-associated control). In embodiments, the cribriform plate image of the normal (non-disease) control subject can be produced as a composite three-dimensional image by imaging cribriform plates of a plurality of normal, healthy control subjects. In embodiments, the cribriform plate image of the disease control can be produced as a composite three-dimensional image by imaging cribriform plates of a plurality of disease-associated subjects, e.g. patients diagnosed with or known to have a neurological disease of an interest such as Alzheimer's Disease. Thus, in embodiments the disease control image is a composite image that was produced by imaging a number of cribriform plates of a plurality of Alzheimer's Disease patients. In embodiments, a control or reference image, i.e. representing a normal (noon-disease) control or a disease control is produced by imaging at least, about or more than 10, 20, 50, 70, 100, 200, 300, 500, 1000 or more than 1000 control subject cribriform plates.

The three-dimensional images of a cribriform plate to be compared can contain three-dimensional images of one or more morphological features of the cribriform plate, which may include, but not limited to, an anterior wall dimension of the cribriform plate, a posterior wall dimension of the cribriform plate (the anterior being closer to the front side of a skull and the posterior being closer to the rear side of the skull when viewed from the top of the skull), a lateral boundary of an olfactory fossa, a length of an olfactory fossa, a width at four locations of an olfactory fossa, a depth of an olfactory fossa, an angle of the posterior wall to the floor of the cribriform plate, a size of an aperture, a branching pattern of an aperture, a position of an aperture, a degree of bone ossification immediately around an aperture, four outer corners of the cribriform plate distance from a midline, margins of a crista galli, a size of a crista galli, or any combination thereof.

In embodiments, the method of the disclosure is performed to detect the presence and/or level of any occluded aperture in the cribriform plate of a subject (or a test subject). In embodiments, the detection is done by comparing the image(s) of the cribriform plate of the test subject to a control image(s) obtained from a disease control subject.

A variety of factors can be considered to detect an occluded aperture from a test subject. For this, an image of cribriform plate of the test sample can be compared to a cribriform plate control image. As described above, the control image can be produced from a single control subject (e.g. a single patient with Alzheimer's Disease). Alternatively, the control image is a composite image produced from a plurality of control subjects (e.g. a number of Alzheimer's Disease patients). For example, an overall shape, a location or position with relation to other elements or in a cribriform plate, a measured value (e.g. length, depth, width, thickness, size, area, diameter, angle, branching pattern of one or more apertures as well as position of aperture in relation to one or more morphological markers that include the four outer corners of the cribriform plate distance from the midline and degree of bone ossification immediately around each aperture) of individual parts of a cribriform plate or the substantially entire cribriform plate can be considered.

Therefore, in embodiments a total number of apertures captured from the cribriform plate image of the test subject is compared to the control image. In embodiments, if the total number of apertures from the image of the test sample is more than or about 5%, about 10%, about 20%, about 30%, about 40%, about 50%, about 60%, about 70%, about 80%, about 90% or about 100% of the total number of apertures from the control image, it can be determined that the occluded aperture(s) is detected in the test sample. In some embodiments, even if the total number of apertures from the image of the test sample is less than 5%, about 10%, about 20%, about 30%, about 40%, about 50%, about 60%, about 70%, about 80%, about 90% or about 100% of the total number of apertures from the control image, it can still be determined that the occluded aperture(s) is detected in the test sample if any other factors such as a size, area and/or depth of one or more apertures is identified comparable to or more than those from the disease control image.

In embodiments, a size of one or more apertures in a cribriform plate can be compared between a test sample and a disease control. Therefore, in embodiments a size of one or more apertures captured from the cribriform plate image of the test subject is compared to the disease control image. In such an example, if the size of one or more apertures from the image of the test sample is more than or about 5%, about 10%, about 20%, about 30%, about 40%, about 50%, about 60%, about 70%, about 80%, about 90% or about 100% of the size of one or more apertures from the control image, it can be determined that the occluded aperture(s) is detected in the test sample. In some embodiments, even if the size of one or more apertures of apertures from the image of the test sample is less than 5%, about 10%, about 20%, about 30%, about 40%, about 50%, about 60%, about 70%, about 80%, about 90% or about 100% of the size of one or more apertures from the disease control image, it can still be determined that the occluded aperture(s) is detected in the test sample if any other factors such as a total number of apertures or an area and/or depth of one or more apertures is identified comparable to or more than those from the disease control image.

In embodiments, an overall area of one or more apertures in a cribriform plate can be compared between a test sample and a disease control. Therefore, in embodiments an overall area of one or more apertures captured from the cribriform plate image of the test subject is compared to the disease control image. In embodiments, if the overall area of one or more apertures from the image of the test sample is more than or about 5%, about 10%, about 20%, about 30%, about 40%, about 50%, about 60%, about 70%, about 80%, about 90% to about 100% of the overall area of one or more apertures from the disease control image, it can be determined that the occluded aperture(s) is detected in the test sample. In some embodiments, even if the overall area of one or more apertures from the image of the test sample is less than 5%, about 10%, about 20%, about 30%, about 40%, about 50%, about 60%, about 70%, about 80%, about 90% or about 100% of that from the disease control image, it can still be determined that the occluded aperture(s) is detected in the test sample if any other factors such as a total number of apertures or a size and/or depth of one or more apertures is identified comparable to or more than those from the disease control image.

In embodiments, a depth of one or more apertures in a cribriform plate can be compared between a test sample and a disease control. Therefore, in embodiments a depth of one or more apertures captured from the cribriform plate image of the test subject is compared to the disease control image. In embodiments, if the depth of one or more apertures from the image of the test sample is more than or about 5%, about 10%, about 20%, about 30%, about 40%, about 50%, about 60%, about 70%, about 80%, about 90% or about 100% of the depth of one or more apertures from the disease control image, it can be determined that the occluded aperture(s) is detected in the test sample. In some embodiments, even if the depth of one or more apertures from the image of the test sample is less than 5%, about 10%, about 20%, about 30%, about 40%, about 50%, about 60%, about 70%, about 80%, about 90% or about 100% of that from the disease control image, it can still be determined that the occluded aperture(s) is detected in the test sample if any other factors such as a total number of apertures or a size and/or area of one or more apertures is identified comparable to or more than those from the disease control image.

In embodiments, a control subject is a normal (non-disease) control subject. Similar to the comparison with a disease control subject, one or more morphological features of the cribriform plate of a test subject can be compared to those of the normal (non-disease) control subject. In embodiments, an overall shape, a location or position with relation to other elements or in a cribriform plate, a measured value (e.g. length, depth, width, thickness, size, area, diameter, angle, branching pattern of one or more apertures as well as position of aperture in relation to one or more morphological markers that include the four outer corners of the cribriform plate distance from the midline and degree of bone ossification immediately around each aperture) of individual parts of a cribriform plate or the substantially entire cribriform plate can be considered. In embodiments, the control image is a composite three-dimensional image that is produced by imaging cribriform plates of a plurality of control subjects, i.e. normal (non-disease) control subjects. In embodiments, the control image is a three-dimensional image that is obtained from a single normal (non-disease) control subject.

In embodiments a total number of apertures captured from the cribriform plate image of the test subject is compared to a normal (non-disease) control image. In such an example, if the total number of apertures from the image of the test sample is more than about 5%, about 10%, about 20%, about 30%, about 40%, about 50%, about 60%, about 70%, about 80%, about 90% or about 100% of the total number of apertures from the normal (non-disease) control image, it can be determined that the occluded aperture(s) is detected in the test sample. In some embodiments, even if the total number of apertures from the image of the test sample is about or less than about 5%, about 10%, about 20%, about 30%, about 40%, about 50%, about 60%, about 70%, about 80%, about 90% or about 100% of the total number of apertures from the normal (non-disease) control image, it can still be determined that the occluded aperture(s) is detected in the test sample if any other factors such as a size, area and/or depth of one or more apertures is identified more than those from the normal (non-disease) control image.

In embodiments, a size of one or more apertures in a cribriform plate can be compared between a test sample and a normal (non-disease) control. Therefore, in embodiments a size of one or more apertures captured from the cribriform plate image of the test subject is compared to the control image. In such an example, if the size of one or more apertures from the image of the test sample is more than about 5%, about 10%, about 20%, about 30%, about 40%, about 50%, about 60%, about 70%, about 80%, about 90% or about 100% of the size of one or more apertures from the normal (non-disease) control image, it can be determined that the occluded aperture(s) is detected in the test sample. In some embodiments, even if the size of one or more apertures of apertures from the image of the test sample is about or less than about 5%, about 10%, about 20%, about 30%, about 40%, about 50%, about 60%, about 70%, about 80%, about 90% or about 100% of the size of one or more apertures from the normal (non-disease) control image, it can still be determined that the occluded aperture(s) is detected in the test sample if any other factors such as a total number of apertures or an area and/or depth of one or more apertures is identified more than those from the normal (non-disease) control image.

In embodiments, an overall area of one or more apertures in a cribriform plate can be compared between a test sample and a normal (non-disease) control. Therefore, in embodiments an overall area of one or more apertures captured from the cribriform plate image of the test subject is compared to the normal (non-disease) control image. In embodiments, if there is more than about 5%, about 10%, about 20%, about 30%, about 40%, about 50%, about 60%, about 70%, about 80%, about 90% or about 100% of the overall area of one or more apertures from the normal (non-disease) control image, it can be determined that the occluded aperture(s) is detected in the test sample. In some embodiments, even if the overall area of one or more apertures from the image of the test sample is about or less than about 5%, about 10%, about 20%, about 30%, about 40%, about 50%, about 60%, about 70%, about 80%, about 90% or about 100% of that from the normal (non-disease) control image, it can still be determined that the occluded aperture(s) is detected in the test sample if any other factors such as a total number of apertures or a size and/or depth of one or more apertures is identified more than those from the normal (non-disease) control image.

In embodiments, a depth of one or more apertures in a cribriform plate can be compared between a test sample and a normal (non-disease) control. Therefore, in embodiments a depth of one or more apertures captured from the cribriform plate image of the test subject is compared to the normal (non-disease) control image. In embodiments, if there is less than about 5%, about 10%, about 20%, about 30%, about 40%, about 50%, about 60%, about 70%, about 80%, about 90% or about 100% of the depth of one or more apertures from the normal (non-disease) control image, it can be determined that the occluded aperture(s) is detected in the test sample. In some embodiments, even if the depth of one or more apertures from the image of the test sample is about or less than about 5%, about 10%, about 20%, about 30%, about 40%, about 50%, about 60%, about 70%, about 80%, about 90% or about 100% of that from the normal (non-disease) control image, it can still be determined that the occluded aperture(s) is detected in the test sample if any other factors such as a total number of apertures or a size and/or area of one or more apertures is identified more than those from the normal (non-disease) control image.

As described above, disruption of cerebrospinal fluid flow across the cribriform plate can predispose the MTG and basal forebrain to accumulate metabolites responsible for the formation of neuritic plaques and neurofibrillary tangles. In embodiments, the disclosure assesses Alzheimer's Disease risk by calculating theoretical cerebrospinal fluid flow capacity (tCFC) through apertures in the cribriform plate. In embodiments, tCFCs produced by the methods of the disclosure can be used for diagnostic purposes and to develop clinical treatment plans for any patient in need. This aspect of the methods provides diagnostic and/or prognostic indicators for Alzheimer's Disease as well as other neurological diseases, e.g., Down syndrome, Parkinson's disease, frontal-temporal dementia, mild cognitive impairment, idiopathic dementia, vascular dementia, amyotrophic lateral sclerosis, Pick's disease, concussive brain injury, supranuclear palsy, Creutzfeld-Jacob disease, normal pressure hydrocephalus, multiple sclerosis.

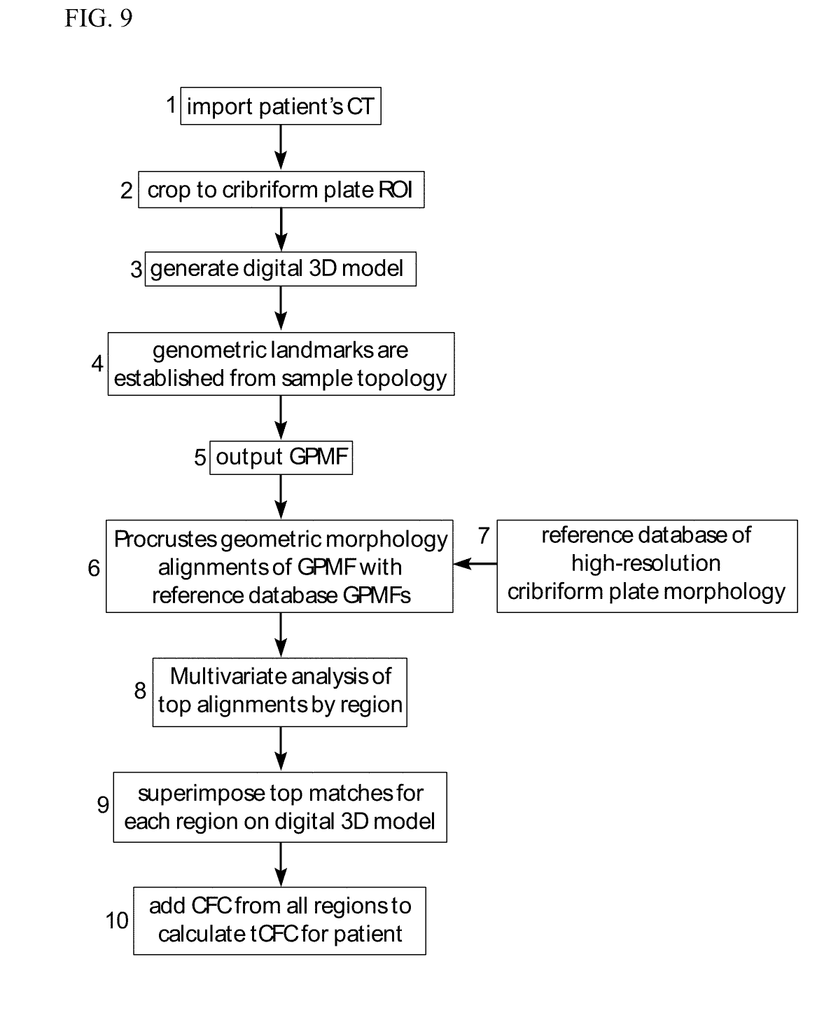

Therefore, in embodiments the methods of the disclosure include measurement of a theoretical cerebrospinal fluid critical flow capacity (tCFC) of a cribriform plate. The calculation can be done by processing the data obtained from scanned images of cribriform plates. A non-limiting and exemplary embodiment of the generation of theoretical cerebrospinal fluid flow capacity (tCFC) in a subject, e.g. a patient is provided in FIG. 9. In embodiments, a clinical CT scan that includes the cribriform plate and surrounding areas is imported. In embodiments, areas outside the region of interest (ROI) are cropped out to reduce file size. In embodiments, image analysis software is used to generate a digital 3D model of the ROI. In embodiments, geometric landmarks are made for surface topology and unusual features are noted, such as gross asymmetry. In embodiments, geometric landmarks are output in a geometric positional map file (GPMF). In embodiments, geometric morphometry is used to align the patient's GPMF with GPMFs in a high-resolution database of cribriform plate morphology, using regional similarities to identify homologous regions from one to many reference cases. In embodiments, multivariate analysis (e.g. PCA and LDA) are used to sort the top alignments from each region of the cribriform plate. In embodiments, matching regions with highest statistical significance are superimposed on the digital 3D model made from the patient's clinical CT scan. In embodiments, theoretical cerebrospinal fluid flow capacity for the cribriform plate is calculated from cerebrospinal fluid channels for each region, using data in the database

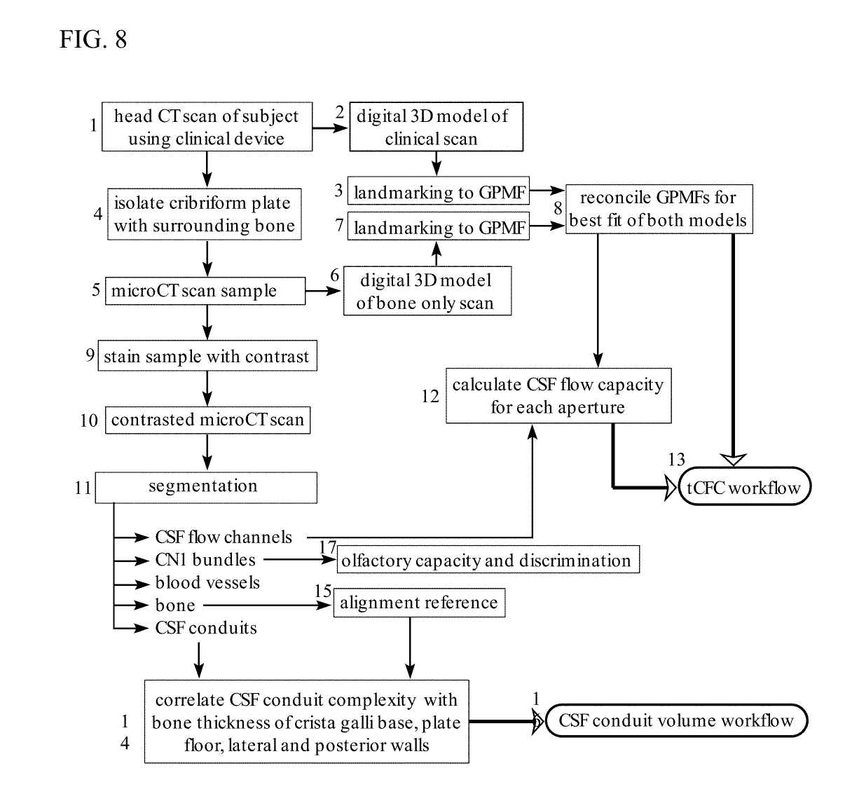

A non-limiting, exemplary embodiment of the database and use of data from the database to measure or calculate a theoretical cerebrospinal fluid critical flow capacity (CFC) is provided in FIG. 8. In embodiments, a head CT scan is collected from a living subject or a post-mortem subject using a clinical grade scanner. In embodiments, the clinical grade scan is used to import image analysis program and process to generate a digital 3D model. In embodiments, geometric landmarks in and around the cribriform plate are noted and output as a geometric positional map file (GPMF). In embodiments, the cribriform plate and surrounding bone are carefully removed from the skull and scanned with a high-resolution microCT scanner. In embodiments, a digital model is made from the microCT scan and geometric landmarks are recorded for output as a GPMF. In embodiments, GFMPs from the clinical and microCT scans are combined using geometric morphometry and differenced are reconciled to produce a GPMF that is a best fit for both. In embodiments, an isolated cribriform plate sample is stained with a contrasting agent, such as metallic-iodine. In embodiments, the contrast-enhanced sample is scanned with microCT. In embodiments, the scan is imported into an image analysis program and segmented into bone, blood vessels, CN1 nerves, cerebrospinal fluid flow channels and cerebrospinal fluid conduits. In embodiments, the location of each aperture is defined by GPMFs and corresponding the cross-sectional area of cerebrospinal fluid flow channels are used to calculate cerebrospinal fluid flow capacity (CFC) for each aperture. In embodiments, GPMFs and CFC values are exported to the tCFC workflow. In embodiments, volumes of cerebrospinal fluid conduits in the base of the crista galli and other regions of the sample are calculated using segmented data that are correlated with local bone thickness calculate relative values in those areas. In embodiments, cerebrospinal fluid conduit correlations are exported to the cerebrospinal fluid conduit volume workflow. In embodiments, values for the cross-sectional area of all CN1 fibers are kept for use in olfactory capacity and discrimination calculations.

In embodiments, a subject that is tested with the methods of the disclosure is an individual, e.g. a patient suspected of having a neurological disease. In embodiments, the subject is a patient suspected of having Alzheimer's Disease. In embodiments, the subject is an individual who is diagnosed with a risk of neurological disease at the time of diagnosis or later. In embodiments, the subject is a human who is not a patient of neurological disease nor diagnosed with a risk of the disease but merely desires to have or benefits from a diagnosis of the disease. In embodiments, a subject is tested with the methods of the disclosure more than once over a course of time, e.g. several months or several years to monitor any changes in the cribriform plate morphology of the subject. This minoring process can allow detection of new development of a neurological disease or any changes in the symptoms caused by an existing disease from a subject or patient.

In one aspect, the disclosure provides a method of diagnosing a subject suspected to having a neurological disease. The method may include (i) imaging the cribriform plate of a subject thereby forming a cribriform plate image and (ii) determining whether the cribriform plate has an occluded aperture based on the cribriform plate image. The presence of the occluded aperture may indicate that the subject has the neurological disease. Therefore, in embodiments where the cribriform plate of a test sample is determined to have an occluded aperture as described above, e.g. based on one or more of comparisons of a total number of apertures, size, area and/or depth of individual apertures and any combinations thereof between cribriform images from a test subject and a control (a disease control and/or a normal (non-disease control), the test subject is considered to be diagnosed with or have a risk of a neurological disease. In embodiments, when the cribriform plate of a test sample is determined to have an occluded aperture, the test sample is considered to be diagnosed with or have a risk of the same disease that the subjects of the disease control image is known to have.

In various embodiments, the methods of the disclosure can be used to diagnose and assess neurological diseases associated with disruptions of cerebrospinal fluid flow and drainage, including but not limited to Alzheimer's Disease, Down syndrome, Parkinson's disease, frontal-temporal dementia, mild cognitive impairment, idiopathic dementia, vascular dementia, Pick's disease, concussive brain injury, supranuclear palsy, Creutzfeld-Jacob disease, normal pressure hydrocephalus, multiple sclerosis, as well as other neurological diseases. In aspects, the neurological diseases associated with disruptions of cerebrospinal fluid flow and drainage is Alzheimer's Disease.

In embodiments, the disclosure provides a method to assess Alzheimer's Disease risk and progression in a subject that may or may not display signs consistent with mild cognitive impairment. In embodiments, the subject is a patient having Alzheimer's Disease. In embodiments, clinical scans from the subject can be used to produce 3D models by comparing them to a database of analyzed cribriform plates. In embodiments, such a database is produced from at least 70 human cribriform plates using high-resolution micro-CT images of excised post-mortem human samples. In embodiments, morphological features are used to generate numerical values for cribriform plate morphology based on dimensions of the olfactory fossa, such as length, width at four locations, depth, and angle of the posterior wall to the floor of the cribriform plate. In embodiments, analysis of the cribriform plate involves annotation of all apertures, including aperture size, shape, angle of descent through the cribriform plate, branching patterns, and position in relation to key morphological markers that include the four outer corners of the cribriform plate distance from the midline and degree of bone ossification immediately around each aperture. In embodiments, morphological features of database 3D models made from micro-CT are refined using a soft-tissue counterstaining method that labeled soft tissues with metallic iodine, followed by micro-CT imaging. In embodiments, contrast-enhanced images resolve soft tissues such at olfactory nerve fibers and bundles, soft tissues surrounding those nerves, blood vessels, and fluid filled chambers, channels and spaces in olfactory bulb, olfactory epithelium and in soft tissues contained within apertures of the cribriform plate. In embodiments, further refinements of database samples are achieved from clinical CT imaging made before cribriform plated were excised from the skull to provide references between low-resolution images that obtained from clinical imaging methods and higher resolution images obtained with micro-CT. In embodiments, the subject's 3D model(s) is/are compared to the database of annotated cribriform plates in two ways: 1) Medical history of the subject is compared to subjects analyzed in the database to find the closest matches to age, sex, height weight, ethnicity, damage sustained to the cribriform plate from traumatic injuries such as broken noses or motor vehicle accidents, and diagnosis of neurological diseases, including a presumptive diagnosis of mild cognitive impairment or Alzheimer's Disease. In embodiments, 3D models from databases subjects with the closest matches are serially aligned with the 3D model from the subject; and 2) Annotations from the subject's 3D models are compared to annotation features from each subject in the databases. In embodiments, results from both modes of comparison are then combined and reconciled to determine how closely the subject's cribriform plate morphology matches one or several subjects from the database. In embodiments, medical information from those database subjects (e.g. controls or references) includes age and onset of neurological diseases including mild cognitive impairment and Alzheimer's Disease. This information can be compared using algorithms that determine risk, prognosis and diagnosis of neurological diseases for the subject.

As described above, certain neurological disease may be associated with an occluded aperture in a cribriform plate which may result in a change of cerebrospinal fluid flow. In particular, the presence or increase of occluded aperture in a subject may result in impeding cerebrospinal fluid flow across the cribriform plate in a subject, leading to the development and/or worsening symptoms of the neurological disease. Therefore, in one aspect the disclosure provides a method of treating a neurological disease in a subject. The method may include (i) imaging the cribriform plate of a subject thereby forming a cribriform plate image, (ii) identifying an occluded aperture based on the cribriform plate image, thereby detecting an occluded aperture in a cribriform plate of a subject, and (iii) draining cerebrospinal fluid from an extracellular compartment above the cribriform plate of the subject.

In embodiments, the drainage of cerebral spinal fluid from a subject who is in need of the treatment performed through the cribriform plate. In embodiments, the drainage is performed by penetrating the cribriform plate with a draining apparatus such as a shunt. In embodiments, the drainage of cerebrospinal fluid is achieved without penetrating the cribriform plate. In embodiments, the drainage without involving the penetration of a cribriform plate can be done via a shunt implanted into the olfactory fossa either from above or through surrounding walls, including the anterior and posterior walls.

In embodiments, the cerebral spinal fluid is drained from the cribriform plate of a subject, e.g. a patient of Alzheimer's Disease to an amount that is effective to the treatment of a neurological disease, e.g. Alzheimer's Disease. This amount, i.e. an effective amount of drainage is determined from the theoretical cerebrospinal fluid critical flow capacity (CFC) that is measured by the method of the disclosure. In embodiments, the CFC is measured (or calculated) from the cribriform plate image of the subject such as Alzheimer's Disease patient and compared to the CFC measured (or calculated) from a control image that is produced from one or more normal, non-disease subjects. In embodiments, if the CFC measured from the subject's image is more than that from the control image, the drainage is done to the amount of difference between the two CFC values such that after the drainage, the patient may have a similar amount of cerebrospinal fluid to the (average amount of) cerebrospinal fluid of the control sample. In embodiments, the amount of drainage is more or less than the difference between the measured CFC values from the subject's image and the control image. In some embodiments, the amount of cerebrospinal fluid drained from the subject, e.g. a patient of Alzheimer's Disease is about 0.5 ml, about 1 ml, about 2 ml, about 3 ml, about 4 ml, about 5 ml, about 6 ml, about 7 ml, about 8 ml, about 9 ml, about 10 ml, about 15 ml, about 20 ml, about 25 ml, about 30 ml, about 35 ml, about 40 ml, about 45 ml, about 50 ml or more per day, or the amount of any intervening values of the foregoing. In embodiments, a device is implanted to a subject, e.g. a patient to drain of an effective amount cerebrospinal fluid from the patient at a certain flow rate. In embodiments, the rate of drainage is set to drain about 0.5 ml, about 1 ml, about 2 ml, about 3 ml, about 4 ml, about 5 ml, about 6 ml, about 7 ml, about 8 ml, about 9 ml, about 10 ml, about 15 ml, about 20 ml, about 25 ml, about 30 ml, about 35 ml, about 40 ml, about 45 ml, about 50 ml or more of cerebrospinal fluid per day from the patient. In embodiments, the device is implanted permanently in the patient to perform the drainage of cerebrospinal fluid.

In embodiments, an effective amount of cerebral spinal fluid drained from a subject is an amount sufficient to accomplish a stated purpose (e.g. achieve the effect for which it is administered, treat a neurological disease, delay onset of the disease and/or reduce one or more symptoms of the disease). An example of the effective amount is an amount sufficient to contribute to the desired treatment that would be sufficient to completely or substantially reduce the symptoms of the disease from the subject. This amount (of drainage of cerebrospinal fluid) can also be referred to as a therapeutically effective amount (of drainage of cerebrospinal fluid). Thus, in some examples, for the given parameter, an effective amount of cerebrospinal fluid drainage will show reduction of the symptoms of a neurological disease (e.g. Alzheimer's Disease) from at least 5%, 10%, 15%, 20%, 25%, 40%, 50%, 60%, 75%, 80%, 90%, or at least 100% from a subject as compared to the level or seriousness of symptoms before the drainage. The exact amounts will depend on the purpose of the treatment, and will be ascertainable by one skilled in the art using known techniques.

The amount and frequency of drainage of cerebral spinal fluid from a subject can vary depending upon a variety of factors, for example, whether the subject suffers from another disease, and its route of drainage; size, age, sex, health, body weight, body mass index, and diet of the subject; nature and extent of symptoms of the disease being treated, kind of concurrent treatment, complications from the disease being treated or other health-related problems. Adjustment and manipulation of amount and frequency may be within the ability of those skilled in the art.

In embodiments, the drainage of cerebral spinal fluid from a subject can be used as a sole therapeutic approach to treat a neurological disease, e.g. Alzheimer's Disease in the subject. In embodiments, the drainage approach can be performed (or administered) in combination with other approaches such as administration of one or more drugs known to be useful in treating a neurological disease.

In embodiments of the methods described herein, the subject is administered a therapeutically effective amount of a neurological agent to treat the neurological disease. The neurological agent can optionally be co-administered with the drainage approach of the disclosure. Exemplary neurological agents include donepezil or a pharmaceutically acceptable salt thereof (available as ARICEPT® from Eisai, Inc.), rivastigmine or a pharmaceutically acceptable salt thereof (available as EXELON® from Novartis), memantine or a pharmaceutically acceptable salt thereof (available as NAMENDA® from Allergan), galantamine or a pharmaceutically acceptable salt thereof (available as RAZADYNE® from Janssen), ergoloid mesylate, combinations of memantine or a pharmaceutically acceptable salt thereof and donepezil or a pharmaceutically acceptable salt thereof (available as NAMZARIC from Allergan), or any combinations thereof. Other examplary neuroglogical agents include deutertrabenazine, cerliponase, amantadine, valbenazine, edaravone, safinamdie, brivaracetam, carbamezepine, eteplirsen, pimavanserin, sumatriptan, nusinsersen, daclizumab, carbidopa/levodopa, carbidopa, levodopa, droxidopa, peginterferon beta-1a, topiramate, eslicarbazepine, perampanel, oxcarbazepine, methylphenidate, clobazam, ezogabine, dalfampridine, onabotulinmtoxin A, glycopyrrolate, clonidine, velaglucerase alfa, interferon beta-1b, interferon beta-1a, vigabatrin, rufinamide, lacosamide, lamotragine, tetrabenazine, cyclobenzaprine, rotigotine, eculizumab, lisdexamfetamine, natalizumab, apomorphine, atomoxetine, dexmethylphenidate, gabapentin, mitoxantrone, oxcarbazepine, zonisamide, levetiracetam, klonopin, aprazolam, glatiramer, pramipexole, ropinirole, divalproex, carbamazepine, tizanidine, riluzole, a pharmaceutically acceptable salt of any one of the foregoing, or a combination of two or more of any of the foregoing. Any drugs or compounds that are known to be helpful or effective in treatment of a neurological disease can be used in combination with the drainage approach of the disclosure.

Utilizing the teachings provided herein, an effective therapeutic treatment regimen can be planned that does not cause substantial toxicity and yet is entirely effective to treat the clinical symptoms demonstrated by the particular patient. This planning should involve the careful choice of therapeutic approach and active compound by considering factors such as potency of the treatment approach, relative bioavailability, patient body weight, presence and severity of adverse side effects, preferred mode of administration, and the toxicity profile of the selected agent or therapeutic approach.

System and Process

In embodiments, the present disclosure provides a processing pipeline that inputs medical imaging files of the cribriform plate, such as a head CT, to produce a digital 3D model of the cribriform plate and surrounding structures, including crista galli and ethmoid bone. In embodiments, automated digital landmarking is performed on the 3D model and output to a geometric positional map file (GPMF). In embodiments, procrustes geometric morphometry alignments of the GPMF with samples in a GPMF referential database are used to calculate tCFC across the cribriform plate. That information can be integrated with the patient's brain size, sex, age, and medical information to generate their probability of developing disorders that could result from insufficient cerebrospinal fluid flow across the cribriform plate, such as Alzheimer's Disease.

In embodiments, the disclosure provides a system and process to assess human cribriform plate morphology in patients that may or may not have mild cognitive impairment. Clinical scans from patients are used to produce digital 3D models for each scan. Automated landmarking of the digital 3D model produces a series of geometric positions that are output to a GPMF. GPMF's taken from scans at different times can be used to evaluate age-dependent effects on cribriform plate morphology. In this instance, the GPMF's are aligned with geometric morphometry without Procrustes adjustments to reveal age-dependent changes in size and shape of the cribriform plate. Statistical analysis of the comparisons is done with multivariate analysis tools such as LDA and PCA.

GPMF's from a patient can also be compared to GPMF's in a referential database that contains many other subjects, including patients with confirmed diagnoses of neurological diseases. The database also includes GFMP's from high-resolution microCT scans of excised post-mortem cribriform plates with and without metal-iodine contrast. Close morphometric matches between from a patient's low resolution clinical scan and cases done with high resolution microCT are used to approximate tissue make up in the patient's cribriform plate. Using cerebrospinal fluid flow capacity calculations from relevant database samples the process produces a theoretical cerebrospinal fluid flow capacity (tCFC) for the patient's cribriform plate. By integrating tCFC with a patient's age, sex, brain volume, and other medical measures, a risk profile is calculated for Alzheimer's Disease and other disorders that may be affected by cerebrospinal fluid flow through the cribriform plate. The processing pipeline continues to evolve as more samples are included in the database, including information derived from each patient analyzed, reducing rates of statistical error.

The system and method also provides a database-guided decision support system based on heterogeneous information sources to assist in the diagnosis, prognosis and treatment of Alzheimer's Disease.

While various embodiments and aspects of the disclosures provided herewith are shown and described herein, it will be obvious to those skilled in the art that such embodiments and aspects are provided by way of example only. Numerous variations, changes, and substitutions will now occur to those skilled in the art without departing from the disclosures. It should be understood that various alternatives to the embodiments of the disclosures described herein may be employed in practicing the invention.

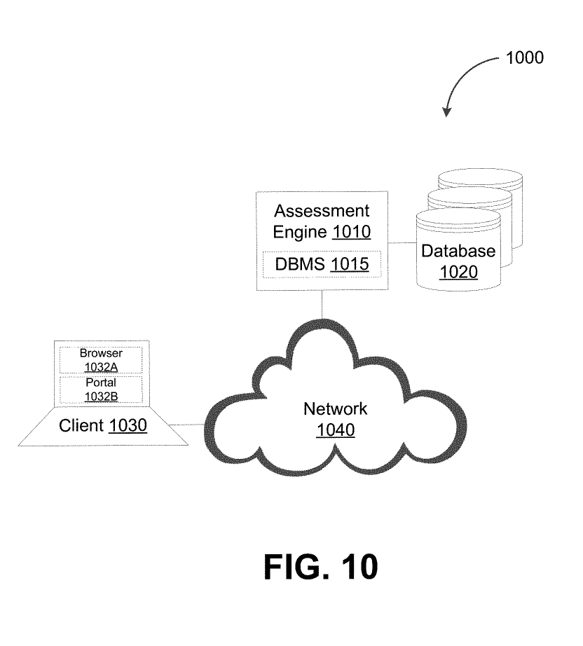

FIG. 10 depicts a system diagram illustrating a system 1000 for assessing human cribriform plate morphology, in accordance with some example embodiments. Referring to FIG. 10, the system 1000 may include an assessment engine 1010 coupled with a database 1020. As shown in FIG. 10, the assessment engine 1110 may include a database management system (DBMS) 1015. The DBMS 1015 may support a variety of operations for accessing the database 1020 including, for example, structured query language (SQL) queries and/or the like. The database 1020 may be any type of database including, for example, an in-memory database, a relational database, a non-SQL (NoSQL) database, and/or the like.

Referring again to FIG. 10, a client 1030 may be communicatively coupled with the assessment engine 1010 via a network 1040. The network 140 may be any wired and/or wireless network including, for example, a wide area network (WAN), a local area network (LAN), a public land mobile network (PLMN), the Internet, and/or the like. In embodiments, one or more functionalities of the assessment engine 1010 may be available as a cloud-based service such as, for example, a software as a service (SaaS) and/or the like. As such, the client 1030 may access the assessment engine 1010 via a web browser 1032A at the client 1030 and/or a dedicated portal 1032B at the client 1030. Alternatively and/or additionally, one or more functionalities of the assessment engine 1010 may be available via a web application accessible through the web browser 1032A at the client 1030.

In embodiments, the database 1020 may store one or more models of human cribriform plates. For example, the database 1020 may store a first model of a human cribriform plate exhibiting a morphology consistent with a subject who is at a first stage of developing a neurological disease such as, for example, Alzheimer's Disease and/or the like. Alternatively and/or additionally, the database 1020 may also store a second model of a human cribriform plate exhibiting a morphology consistent with a subject who is at a second stage of developing the neurological disease.

The models of human cribriform plates stored at the database 1020 may be digital 3D models formed based on images of cribriform plates including, for example, CT scans, MRI scans, PET scans, ultrasound images, far-red imaging scans, and/or the like. Furthermore, these models may be composite models formed by combining different models of human cribriform plates originating from multiple subjects. For instance, two or more models of cribriform plates may be combined by identifying consistent apertures that are present in all of the models and aligning the models based on these consistent apertures. Alternatively and/or additionally, two or more models of cribriform plates may be combined by identifying incongruent apertures that are present in some but not all of the models. The two or more models may be aligned based on the incongruent apertures instead or and/or in addition to the consistent apertures. It should be appreciated that the consistent apertures and the incongruent apertures may be weighted when two or more models are aligned based on both consistent apertures and incongruent apertures. For instance, the incongruent apertures may be assigned lesser weights relative to the consistent apertures.

In embodiments, the client 1030 may access the assessment engine 1010 in order to determine whether a patient has a neurological disease such as, for example, Alzheimer's Disease and/or the like. For instance, the client 1030 may submit, to the assessment engine 1010, a third model of the cribriform plate of the patient. In response, the assessment engine 1010 may query the database 1020 to identify one or more of the models stored at the database 1020 that match the third model. For example, to match the third model to the first model and/or the second model, the assessment engine 1010 may align the apertures that are present in each of the first model, the second model, and/or the third model.

In embodiments, the assessment engine 1010 may determine whether the patient has the neurological disease based on the third model of the patient's cribriform plate being matched to the first model and/or the second model stored at the database 1020. For example, in the event that the third model of the patient's cribriform plate is matched to the first model, the assessment engine 1010 may identify occluded apertures present in the cribriform plate of the patient based on the occluded apertures present in the first model. Alternatively and/or additionally, if the third model of the patient's cribriform plate is matched to the second model, the assessment engine 1010 may identify occluded apertures present in the cribriform plate of the patient based on the occluded apertures present in the second model. The presence of one or more occluded apertures in the cribriform plate of the patient may affect the cerebrospinal fluid critical flow capacity of the patient. For instance, based on the occluded apertures present in the cribriform plate of the patient, the assessment engine 1010 may determine the cerebrospinal fluid critical flow capacity of the patient. The assessment engine 1010 may further determine that the patient has the neurological disease when the cerebrospinal fluid critical flow capacity fails to exceed a threshold value.

In embodiments, the assessment engine 1010 may also determine a stage of development for the neurological disease the third model of the patient's cribriform plate being matched to the first model and/or the second model stored at the database 1020. As noted, the first model may correspond to a human cribriform plate exhibiting a morphology consistent with a subject who is at a first stage of developing a neurological disease. Meanwhile, the second model may correspond to a human cribriform plate exhibiting a morphology consistent with a subject who is at a second stage of developing the neurological disease. Thus, the assessment engine 1010 may determine that the patient is at the first stage of developing the neurological disease if the third model of the patient's cribriform plate matches the first model. Alternatively and/or additionally, the assessment engine 1010 may determine that the patient is at the second stage of developing the neurological disease if the third model of the patient's cribriform plate matches the second model.

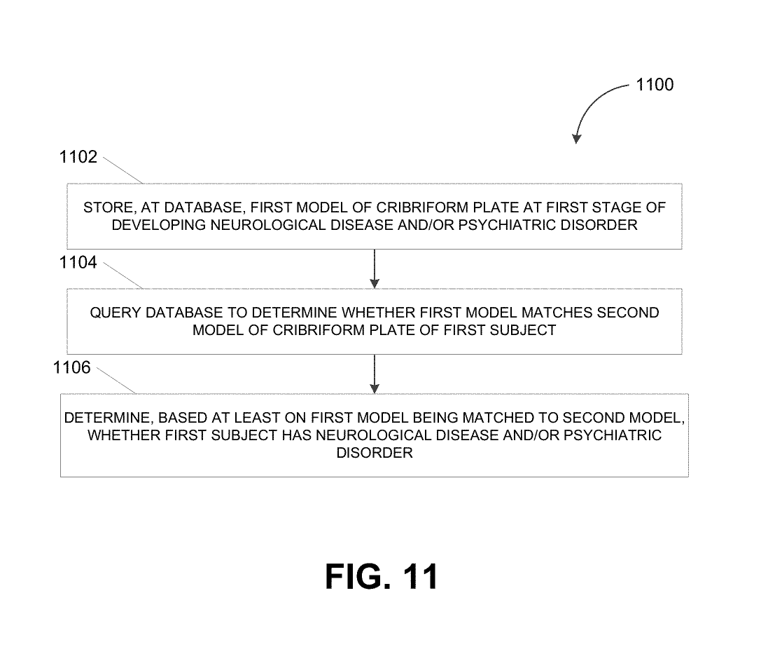

FIG. 11 depicts a flowchart illustrating a process 1100 for assessing human cribriform plate morphology, in accordance with some example embodiments. Referring to FIG. 10 and FIG. 11, the process 1100 may be performed by the assessment engine 1010.

The assessment engine 1010 may store, at the database 1020, a first model of a cribriform plate a first stage of developing a neurological disease (1102). For example, the assessment engine 1010 may store, at the database 1020, a plurality of models of cribriform plates including, for example, the first model of a human cribriform plate exhibiting a morphology consistent with a subject who is at a first stage of developing the neurological disease and the second model of a human cribriform plate exhibiting a morphology consistent with a subject who is at a second stage of developing the neurological disease. As noted, the models stored at the database 1020 may be 3D models formed based on images of cribriform plates including, for example, CT scans, MRI scans, PET scans, ultrasound images, far-red imaging scans, and/or the like. Furthermore, the models stored at the database 1020 may be composite models formed by combining different models of human cribriform plates originating from multiple subjects.

The assessment engine 1010 may query the database 1020 to determine whether the first model matches a second model of a cribriform plate of a first subject (1104). As noted, the client 1030 may submit the third model of the patient's cribriform plate. The assessment engine 1010 may respond by matching the third model of the patient's cribriform plate to one or more of the plurality of models of cribriform plates (e.g., the first model and/or the second model) stored at the database 1020. For example, the assessment engine 1010 may query the database 1020. Furthermore, the assessment engine 1010 may determine whether the third model of the patient's cribriform plate matches one or more of the plurality of cribriform plates stored at the database 1020 by aligning the apertures that are present in the models.

The assessment engine 1010 may determine whether the first subject has the neurological disease based at least on the first model being matched to the second model (1106). For example, the assessment engine 1010 may determine whether the patient has the neurological disease based on the third model of the patient's cribriform plate being matched to the first model and/or the second model stored at the database 1020. If the third model of the patient's cribriform plate is matched to the first model, the assessment engine 1010 may identify occluded apertures present in the cribriform plate of the patient based on the occluded apertures present in the first model. Alternatively and/or additionally, if the third model of the patient's cribriform plate is matched to the second model, the assessment engine 1010 may identify occluded apertures present in the cribriform plate of the patient based on the occluded apertures present in the second model. In embodiments, the assessment engine 1010 may determine the cerebrospinal fluid critical flow capacity of the patient based on the occluded apertures present in the cribriform plate of the patient. Furthermore, the assessment engine 1010 may determine that the patient has the neurological disease when the cerebrospinal fluid critical flow capacity fails to exceed a threshold value.

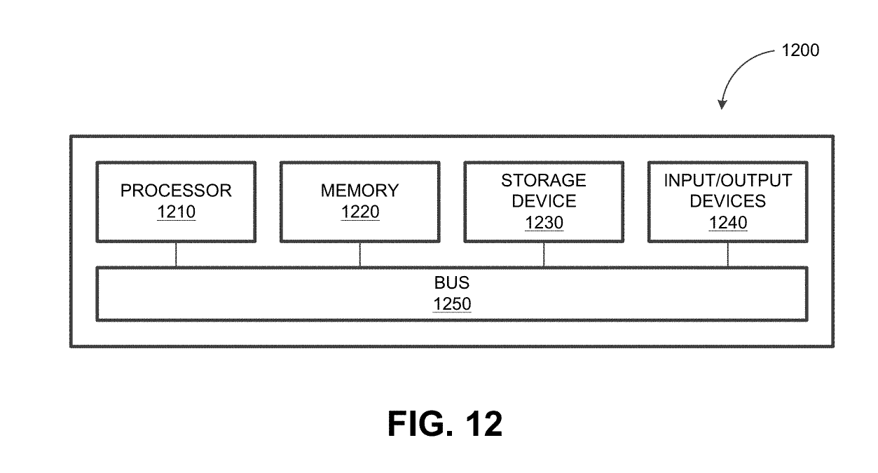

FIG. 12 depicts a block diagram illustrating a computing system 1200, in accordance with some example embodiments. Referring to FIGS. 10-12, the computing system 1200 may implement the assessment engine 1010 and/or any component therein.

As shown in FIG. 5, the computing system 1200 can include a processor 1210, a memory 1220, a storage device 1230, and input/output devices 1240. The processor 1210, the memory 1220, the storage device 1230, and the input/output devices 1240 can be interconnected via a system bus 1250. The processor 1210 is capable of processing instructions for execution within the computing system 1200. Such executed instructions can implement one or more components of, for example, the database system 100 and/or the multitenant database system 200. In some example embodiments, the processor 1210 can be a single-threaded processor. Alternately, the processor 1210 can be a multi-threaded processor. The processor 1210 is capable of processing instructions stored in the memory 1220 and/or on the storage device 1230 to display graphical information for a user interface provided via the input/output device 1240.

The memory 1220 is a computer readable medium such as volatile or non-volatile that stores information within the computing system 1200. The memory 1220 can store data structures representing configuration object databases, for example. The storage device 1230 is capable of providing persistent storage for the computing system 1200. The storage device 1230 can be a floppy disk device, a hard disk device, an optical disk device, or a tape device, or other suitable persistent storage means. The input/output device 1240 provides input/output operations for the computing system 1200. In some example embodiments, the input/output device 1240 includes a keyboard and/or pointing device. In various implementations, the input/output device 1240 includes a display unit for displaying graphical user interfaces.

According to some example embodiments, the input/output device 1240 can provide input/output operations for a network device. For example, the input/output device 1240 can include Ethernet ports or other networking ports to communicate with one or more wired and/or wireless networks (e.g., a local area network (LAN), a wide area network (WAN), the Internet).

In some example embodiments, the computing system 1200 can be used to execute various interactive computer software applications that can be used for organization, analysis and/or storage of data in various formats. Alternatively, the computing system 1200 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities (e.g., SAP Integrated Business Planning as an add-in for a spreadsheet and/or other type of program) or can be standalone computing products and/or functionalities. Upon activation within the applications, the functionalities can be used to generate the user interface provided via the input/output device 1240. The user interface can be generated and presented to a user by the computing system 1200 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

### EMBODIMENTS

### Embodiment 1

A method of detecting an occluded aperture in a cribriform plate of a subject, the method comprising: (i) imaging the cribriform plate of a subject thereby forming a cribriform plate image; (ii) identifying an occluded aperture based on the cribriform plate image, thereby detecting an occluded aperture in the cribriform plate of the subject

### Embodiment 2

The method of Embodiment 1, wherein the identifying step comprises comparing the cribriform plate image to a cribriform plate control image.

### Embodiment 3

The method of Embodiment 2, wherein the cribriform plate control image is a composite three-dimensional image.

### Embodiment 4

The method of Embodiment 3, wherein the composite three-dimensional image is produced by imaging a plurality of subject cribriform plates.

### Embodiment 5

The method of Embodiment 4, wherein the plurality of subject cribriform plates is more than one thousand subject cribriform plates.

### Embodiment 6

The method of Embodiment 4, wherein the plurality of subject cribriform plates is more than one hundred thousand subject cribriform plates.

### Embodiment 7

The method of any one of Embodiments 1 to 6, wherein the identifying step further comprises identifying a plurality of occluded apertures based on the cribriform plate image.

### Embodiment 8

The method of Embodiment 7, wherein the identifying comprises measuring the area of the occluded aperture and the plurality of occluded apertures.

### Embodiment 9

The method of Embodiment 7, wherein the identifying comprises measuring a theoretical cerebrospinal fluid critical flow capacity of the occluded aperture and the plurality of occluded apertures.

### Embodiment 10

The method of any one of Embodiments 1 to 9, wherein the subject has or is suspected of having a neurological disease.

### Embodiment 11

The method of Embodiment 10, wherein the neurological disease is Alzheimer's Disease, Down syndrome, Parkinson's disease, Pick's disease, dementia, a brain injury, supranuclear palsy, Creutzfeldt-Jakob disease, normal pressure hydrocephalus, multiple sclerosis, Huntington's Disease, amyotrophic lateral sclerosis, muscular dystrophy, epilepsy, autism, or cerebral palsy. In aspects, the neurological disese of Embodiment 10 is Alzheimer's disease. In aspects, the neurological disese of Embodiment 10 is Down Syndrome. In aspects, the neurological disese of Embodiment 10 is a brain injury. In aspects, the neurological disese of Embodiment 10 is dementia. In aspects, the neurological disese of Embodiment 10 is dementia, wherein the dementia is a mild cognitive impairment.

### Embodiment 12

The method of any one of Embodiments 1 to 11, wherein the cribriform plate image is a computed tomography cribriform plate image, magnetic resonance imaging cribriform plate image, positron emission tomography cribriform plate image, ultrasound cribriform plate image, or far-red imaging cribriform plate image.

### Embodiment 13

A method of diagnosing a subject suspected of having a neurological disease, the method comprising: (i) imaging the cribriform plate of a subject thereby forming a cribriform plate image; (ii) determining whether the cribriform plate comprises an occluded aperture based on the cribriform plate image, wherein the presence of the occluded aperture indicates the subject has the neurological disease

### Embodiment 14

The method of Embodiment 13, wherein the cribriform plate control image is a composite three-dimensional image.

### Embodiment 15

The method of Embodiment 14, wherein the composite three-dimensional image is produced by imaging a plurality of subject cribriform plates.

### Embodiment 16

The method of Embodiment 15, wherein the plurality of subject cribriform plates is more than one thousand subject cribriform plates.

### Embodiment 17

The method of Embodiment 15, wherein the plurality of subject cribriform plates is more than one hundred thousand subject cribriform plates.

### Embodiment 18

The method of any one of Embodiments 13 to 17, wherein the determining step comprises comparing the cribriform plate image to a cribriform plate control image.

### Embodiment 19

The method of any one of Embodiments 13 to 18, wherein the determining step further comprises identifying a plurality of occluded apertures based on the cribriform plate image.

### Embodiment 20

The method of Embodiment 19, wherein the determining comprises measuring the area of the occluded aperture and the plurality of occluded apertures.

### Embodiment 21

The method of Embodiment 19, wherein the determining comprises measuring a theoretical cerebrospinal fluid critical flow capacity of the occluded aperture and the plurality of occluded apertures.

### Embodiment 22

The method of any one of Embodiments 13 to 21, wherein the subject has or is suspected of having Alzheimer's Disease, Down syndrome, Parkinson's disease, Pick's disease, dementia, a brain injury, supranuclear palsy, Creutzfeldt-Jakob disease, normal pressure hydrocephalus, multiple sclerosis, Huntington's Disease, amyotrophic lateral sclerosis, muscular dystrophy, epilepsy, autism, or cerebral palsy. In aspects, the neurological disese of any one of Embodiments 13 to 21 is Alzheimer's disease. In aspects, the neurological disese of Embodiments 13 to 21 is Down Syndrome. In aspects, the neurological disese of Embodiments 13 to 21 is a brain injury. In aspects, the neurological disese of Embodiments 13 to 21 is dementia. In aspects, the neurological disese of Embodiments 13 to 21 is dementia, wherein the dementia is a mild cognitive impairment.

### Embodiment 23

The method of any one of Embodiments 13 to 22, wherein the cribriform plate image is a computed tomography cribriform plate image, magnetic resonance imaging cribriform plate image, positron emission tomography cribriform plate image, ultrasound cribriform plate image, or far-red imaging cribriform plate image.

### Embodiment 24

A method of treating a neurological disease in a subject, the method comprising: (i) imaging the cribriform plate of a subject thereby forming a cribriform plate image; (ii) identifying an occluded aperture based on the cribriform plate image, thereby detecting an occluded aperture in a cribriform plate of a subject; and draining cerebrospinal fluid from an extracellular compartment above the cribriform plate of the subject.

### Embodiment 25

The method of Embodiment 24, wherein the identifying step comprises comparing the cribriform plate image to a cribriform plate control image.

### Embodiment 26

The method of Embodiment 25, wherein the cribriform plate control image is a composite three-dimensional image.

### Embodiment 27

The method of Embodiment 26, wherein the composite three-dimensional image is produced by imaging a plurality of subject cribriform plates.

### Embodiment 28

The method of Embodiment 27, wherein the plurality of subject cribriform plates is more than one thousand subject cribriform plates.

### Embodiment 29

The method of Embodiment 27, wherein the plurality of subject cribriform plates is more than one hundred thousand subject cribriform plates.

### Embodiment 30

The method of any one of Embodiments 24 to 29, wherein the identifying step further comprises identifying a plurality of occluded apertures based on the cribriform plate image.

### Embodiment 31

The method of Embodiment 30, wherein the identifying comprises measuring the area of the occluded aperture and the plurality of occluded apertures.

### Embodiment 32

The method of Embodiment 30, wherein the identifying comprises measuring a theoretical cerebrospinal fluid critical flow capacity of the occluded aperture and the plurality of occluded apertures.

### Embodiment 33

The method of any one of Embodiments 24 to 32, wherein the subject has or is suspected of having a neurological disease.

### Embodiment 34

The method of Embodiment 33, wherein the neurological disease is Alzheimer's Disease, Down syndrome, Parkinson's disease, Pick's disease, dementia, a brain injury, supranuclear palsy, Creutzfeldt-Jakob disease, normal pressure hydrocephalus, multiple sclerosis, Huntington's Disease, amyotrophic lateral sclerosis, muscular dystrophy, epilepsy, autism, or cerebral palsy. In aspects, the neurological disese of Embodiment 33 is Alzheimer's disease. In aspects, the neurological disese of Embodiment 33 is Down Syndrome. In aspects, the neurological disese of Embodiment 33 is a brain injury. In aspects, the neurological disese of Embodiment 33 is dementia. In aspects, the neurological disese of Embodiment 33 is dementia, wherein the dementia is a mild cognitive impairment.

### Embodiment 35

The method of any one of Embodiments 24 to 34, wherein the cribriform plate image is a computed tomography cribriform plate image, magnetic resonance imaging cribriform plate image, positron emission tomography cribriform plate image, ultrasound cribriform plate image, or far-red imaging cribriform plate image.

### Embodiment 36

The method of any one of Embodiments 24 to 35, wherein the draining is performed through the cribriform plate.

### Embodiment 37

The method of Embodiment 36, wherein further comprising penetrating the cribriform plate with a draining apparatus.

### Embodiment 38

The method of Embodiment 37, wherein the draining apparatus is a shunt.

### Embodiment 39

A system, comprising: (i) at least one data processor; and (ii) at least one memory including instructions which, when executed by the at least one data processor, result in operations comprising: (a) storing, in a database, a first model of a cribriform plate at a first stage of developing a neurological disease; (b) querying the database to determine whether the first model matches a second model of a cribriform plate of a first subject; and (c) determining, based at least on the first model being matched to the second model, whether the first subject has the neurological disease.

### Embodiment 40

The system of Embodiment 39, wherein the first model is matched to the second model by at least aligning one or more apertures present in each of the first model and the second model.

### Embodiment 41

The system of Embodiment 40, wherein the determination of whether the first subject has the neurological disease comprises: (i) identifying, based at least on one or more occluded apertures present in the first model, at least one occluded aperture in the cribriform plate of the first subject; (ii) determining, based on the at least one occluded aperture present in the cribriform plate of the first subject, a cerebrospinal fluid critical flow capacity for the first subject; and (iii) determining that the first subject has the neurological disease based at least on the cerebrospinal fluid critical flow capacity being less than a threshold value.

### Embodiment 42

The system of Embodiment 39, 40, or 41, further comprising storing, at the database, a third model of a cribriform plate at a second stage of developing the neurological disease.

### Embodiment 43

The system of Embodiment 42, further comprising in response to the first model being matched to the second model, determining that the first subject is at the first stage of developing the neurological disease; and in response to the third model being matched to the second model, determining that the first subject is at the second stage of developing the neurological disease.

### Embodiment 44

The system of any one of Embodiments 39 to 43, further comprising generating the first model, the second model, or the first model and the second model based on a plurality of cribriform plate images.

### Embodiment 45

The system of Embodiment 44, where in the plurality of cribriform plate images comprise one or more computed tomography images, magnetic resonance imaging images, positron emission tomography images, ultrasound images, far-red imaging images, or a combination of two or more thereof.

### Embodiment 46

The system of any one of Embodiments 39 to 45, wherein the first model comprises composite model generated by at least combining a third model of a cribriform plate of a second subject and a fourth model of a cribriform plate of a third subject, and wherein the second subject and the third subject are both at the first stage of developing the neurological disease.

### Embodiment 47

The system of Embodiment 46, wherein the third model and the fourth model are combined by at least identifying one or more consistent apertures between the third model and the fourth model; and aligning, based at least on the one or more consistent apertures, the third model and the fourth model.

### Embodiment 48

The system of Embodiment 47, wherein the combining of the third model and the fourth model further comprises identifying one or more incongruent apertures between the third model and the fourth model; and aligning, based at least on the one or more incongruent apertures, the third model and the fourth model.

### Embodiment 49

The system of Embodiment 48, wherein the third model and the fourth model are aligned by at least weighting the one or more consistent apertures and the one or more incongruent apertures, and wherein the one or more incongruent apertures are assigned a lesser weight relative to the one or more consistent apertures.

### Embodiment 50

The system of any one of Embodiments 39 to 49, wherein the first model, the second model, or the first model and the second model comprise three-dimensional models.

### Embodiment 51

The system of any one of Embodiments 39 to 50, wherein the neurological disease is Alzheimer's Disease, Down syndrome, Parkinson's disease, Pick's disease, dementia, a brain injury, supranuclear palsy, Creutzfeldt-Jakob disease, normal pressure hydrocephalus, multiple sclerosis, Huntington's Disease, amyotrophic lateral sclerosis, muscular dystrophy, epilepsy, autism, or cerebral palsy. In aspects, the neurological disese of any one of Embodiments 39 to 50 is Alzheimer's disease. In aspects, the neurological disese of Embodiments 39 to 50 is Down Syndrome. In aspects, the neurological disese of Embodiments 39 to 50 is a brain injury. In aspects, the neurological disese of Embodiments 39 to 50 is dementia. In aspects, the neurological disese of Embodiments 39 to 50 is dementia, wherein the dementia is a mild cognitive impairment.

### EXAMPLES

### Example 1: Prognosis of Neurological Diseases

The method is used as a prognostic indicator of neurological disease from a population of aging patients using annual or semi-annual CT imaging of the cribriform plate and surrounding bones. As part of annual medical or dental exams patients have their heads and/or maxilla scanned, including the cribriform plate area, every year or two beginning at age 50. The processing pipeline produces a 3D model from each patient's scan and automatically positions geometric markers using established landmarks and unusual features, which is output to a GPMF. Geometric morphometry is used to align all GPMF's from a patient to gauge age-related changes in cribriform plate morphology, including trends such as growth of a bony veil down the posterior wall of the olfactory fossa. To overcome low-resolution limitations of clinical grade CT scanners, GPMF's are aligned with a referential database of high-resolution microCT images to approximate of aperture openings, occlusion, and tCFC. LDA is used to estimate when cribriform plate occlusion will reach a threshold beyond which cerebrospinal fluid-mediated clearance of the MTG and basal forebrain is unable to clear insoluble metabolites that trigger Alzheimer's Disease pathology. These methods can be used to monitor cribriform plate morphologies in aging human populations of any size to plan for medical care, prophylactic interventions, and other issues.

### Example 2: Assessment of Alzheimer's Disease Risk

The method is used to assess Alzheimer's Disease (AD) risk in a patient with idiopathic dysosmia and a family history of Alzheimer's Disease. The patient's mother developed Alzheimer's Disease in her late 60's and his father developed Alzheimer's Disease with Lewy Body Dementia at in his early 70's. A recent head CT is used to produce a 3D model, generate landmarks and produce a GPMF. The processing pipeline uses geometric morphometrics to compare the patient's GPMF to samples in a GPMF database having cribriform plate models produced by high-resolution microCT analysis of patients with confirmed post-mortem diagnoses. The method identifies morphological characteristics of note, including but bony veil on the posterior wall of the olfactory fossa, regions of extensive ossification, and asymmetry. For example, the processing pipeline may indicate extensive occlusion of cribriform plate apertures that are consistent with mild cognitive impairment leading to Alzheimer's Disease in the 6^{th} decade of life. Alzheimer's Disease risk and prognosis are calculated from tCFC, age, sex, height, weight, brain volume, memory test results, family history and other relevant medical measures. These results indicate the patient is a good candidate for therapeutic intervention(s) to increase cerebrospinal fluid drainage from the olfactory fossa.

### Example 3: Prognosis in a Patient with a History of Head Trauma

The method is used to evaluate cribriform structure as a prognostic indicator in a patient with a history of head trauma. Ten years earlier a military veteran suffered traumatic head injury in the field of battle and was treated for a concussion, broken nose and rhinal cerebrospinal fluid leakage syndrome, resulting from proximity to an improvised explosive device. Treatment included surgical application of fibrin glue to seal cerebrospinal fluid leakage through a fractured cribriform plate, and external setting of the broken nose. Recently, the patient began to show signs of mild cognitive impairment (MCI). The patient's clinicians ordered a head CT that included the cribriform plate, which was provided along with a head CT taken at the time of the field injury. The processing pipeline of the disclosure produced 3D models from these two time points and automatically placed position markers at landmark locations and unusual features and output as a GPMF. Age-related changes between the two scans that occurred over the past 10 years are determined by aligning both GPMF's and multivariate analysis done by PCA. Findings included an uneven cribriform plate, which occurs in broken due to deviation of the perpendicular plate of the ethmoid bone, which is contiguous with base of the crista galli. Deviation of the perpendicular plate to the left causes deviation of the crista galli with a chance of cracking one side of the cribriform plate and compressing the other side. This asymmetry is not corrected by external setting of the nose, which allows the injured side of the cribriform plate to heal in an elevated position. In studies that form the basis of this patent, extensive ossification usually occurs on the elevated side of asymmetrical cribriform plates that were likely the result of nose injuries (see FIG. 3), and may predispose the individual to Alzheimer's Disease pathology. Next, geometric morphology is used to align GPMF's from the more recent scan to GPMF's in a referential database of high-resolution microCT scans of cribriform plates isolated from subjects with post-mortem pathology. Approximate measures are calculated for aperture cross-sectional areas and tCFC using LDA multivariate analysis. Diagnostic and prognostic values of the known cases are used to determine the patient's risk of developing Alzheimer's Disease and other neurological diseases that may be impacted by cribriform plate morphology.

### Example 4: Evaluation of Cribriform Plate Dynamics in a Subject with Down Syndrome

The method is used to evaluate cribriform plate dynamics in an 18-year-old (18 y) male with Down syndrome (trisomy 21), who presents with dysosmia. A recent study of Down syndrome patients showed significant dysosmia or anosmia in all adult subjects, while an earlier study found no significant problems with olfaction in children with Down syndrome. A characteristic facial feature of Down syndrome patients is a pug nose indicative of brachycephaly, which involves malformation of facial bones that include the ethmoid and cribriform plate. Importantly, nearly all Down syndrome patients develop Alzheimer's Disease pathology in the 4^{th} or 5^{th} decade of life. Changes to facial bones that occur during puberty may adversely affect cribriform plate morphology and restrict cerebrospinal fluid outflow. The method is used to process a head CT from the patient, generating a digital 3D model, automatic landmarking, and generation of a GPMF. The method determines the degree of aperture porosity, and olfactory fossa shape. In this case, an earlier dental CT that included the cribriform plate was taken at age 10 after a bicycle accident. Alignment of GPMF's from both ages are used to approximate puberty-related changes that will be included in the referential database. Analysis reveals significant occlusion of the patient's cribriform plate and a low tCFC that warrants invention to increase cerebrospinal fluid-mediated clearance of the MTG and basal forebrain.

### Example 5: Diagnosis in Patient with Mild Cognitive Impairment

The method is used to evaluate cribriform plate dynamics in a patient with mild cognitive impairment. The patient undergoes CT imaging of her head with emphasis on the cribriform plate. Scan images are processed with filters that may or may not include digital tools such as Gaussian blur and smoothing to produce a resolved 3D image of cribriform plate bony structure. The 3D image is then annotated with positional markers for features that include anterior and posterior walls, apertures, regions of ossification, asymmetry, lateral boundaries of the olfactory fossa, margins and size of the crista galli, and overall dimensions of the floor of the cribriform plates. The patient's annotated 3D image is then digitally mapped to a database of cribriform plates consisting of high-resolution micro-CT scans and clinical CT scans based on but not limited to annotated features in the patient's cribriform plate and the database. Cases in the database are identified that have the closest matches to the patient's age, sex, ethnicity, genomic information, and medical history including putative or confirmed diagnoses of Alzheimer's Disease, mild cognitive impairment or other neurological diseases. Cribriform plates from those cases are then individually mapped to morphological features of the patient's 3D image to create a second set of comparison measures. Comparisons and reconciliation of differences between the most similar cases identified in both searches is then performed to calculate prognostic probabilities for dementia, mild cognitive impairment or other neurological diseases. The results of this analysis can be used in the development of care and treatment plans for the patients.

### REFERENCE

Ethell, D W, “Disruption of cerebrospinal fluid flow through the olfactory system may contribute to AD pathogenesis,” Journal of Alzheimers Disease 451(4):1021-30, 2014

What is claimed is:

1. A method of detecting an occluded aperture in a cribriform plate of a subject, the method comprising:
(i) imaging the cribriform plate of a subject thereby forming a cribriform plate image;
(ii) identifying an occluded aperture based on the cribriform plate image, thereby detecting an occluded aperture in the cribriform plate of the subject.

2. The method of claim 1, wherein the identifying step comprises comparing the cribriform plate image to a cribriform plate control image.

3. The method of claim 2, wherein the cribriform plate control image is a composite three-dimensional image.

4. The method of claim 3, wherein the composite three-dimensional image is produced by imaging a plurality of subject cribriform plates.

5. The method of claim 1, wherein the identifying step further comprises identifying a plurality of occluded apertures based on the cribriform plate image.

6. The method of claim 6, wherein the identifying comprises measuring the area of the occluded aperture and the plurality of occluded apertures.

7. The method of claim 1, wherein the cribriform plate image is a computed tomography cribriform plate image, magnetic resonance imaging cribriform plate image, positron emission tomography cribriform plate image, ultrasound cribriform plate image, or far-red imaging cribriform plate image.

8. The method of claim 1, further comprising: (i) draining cerebrospinal fluid from an extracellular compartment above the cribriform plate of the subject; (ii) administering a therapeutically effective amount of a neurological agent to the subject, or (iii) a combination of (i) and (ii).

9. A method of diagnosing a subject suspected of having a neurological disease, the method comprising:
(i) imaging the cribriform plate of a subject thereby forming a cribriform plate image;
(ii) determining whether the cribriform plate comprises an occluded aperture based on the cribriform plate image, wherein the presence of the occluded aperture indicates the subject has the neurological disease.

10. The method of claim 9, further comprising: (i) draining cerebrospinal fluid from an extracellular compartment above the cribriform plate of the subject; (ii) administering a therapeutically effective amount of a neurological agent to the subject, or (iii) a combination of (i) and (ii).

11. A method of treating a neurological disease in a subject, the method comprising:
(i) imaging the cribriform plate of a subject thereby forming a cribriform plate image;
(ii) identifying an occluded aperture based on the cribriform plate image, thereby detecting an occluded aperture in a cribriform plate of a subject; and
(iii) draining cerebrospinal fluid from an extracellular compartment above the cribriform plate of the subject.

12. The method of claim 11, wherein the identifying step comprises comparing the cribriform plate image to a cribriform plate control image.

13. The method of claim 12, wherein the cribriform plate control image is a composite three-dimensional image.

14. The method of claim 13, wherein the composite three-dimensional image is produced by imaging a plurality of subject cribriform plates.

15. The method claim 11, wherein the identifying step further comprises identifying a plurality of occluded apertures based on the cribriform plate image.

16. The method of claim 16, wherein the identifying comprises measuring the area of the occluded aperture and the plurality of occluded apertures.

17. The method of claim 16, wherein the identifying comprises measuring a theoretical cerebrospinal fluid critical flow capacity of the occluded aperture and the plurality of occluded apertures.

18. The method of claim 11, wherein the cribriform plate image is a computed tomography cribriform plate image, magnetic resonance imaging cribriform plate image, positron emission tomography cribriform plate image, ultrasound cribriform plate image, or far-red imaging cribriform plate image.

19. The method of claim 11, further comprising administering a therapeutically effective amount of a neurological agent to the subject.

20. A system, comprising:
(i) at least one data processor; and
(ii) at least one memory including instructions which, when executed by the at least one data processor, result in operations comprising:
(a) storing, in a database, a first model of a cribriform plate at a first stage of developing a neurological disease;
(b) querying the database to determine whether the first model matches a second model of a cribriform plate of a first subject; and
(c) determining, based at least on the first model being matched to the second model, whether the first subject has the neurological disease.
